(19) United States
(12) Patent Application Publication
(10) Pub. No.: US 2026/0216349 A1
(43) Pub. Date: Jul. 30, 2026

(54) STABILIZED BIOPOLYMER COMPOSITION, THEIR MANUFACTURE AND USE

(71) Applicant: BASE SE, Ludwigshafen am Rhein (DE)

(72) Inventors: Meike Maria ROSKAMP, Ludwigshafen am Rhein (DE); Sophia EBERT, Ludwigshafen am Rhein (DE); Matthias KELLERMEIER, Ludwigshafen am Rhein (DE); Nadine LOEW, Ludwigshafen am Rhein (DE)

(21) Appl. No.: 19/149,112

(22) PCT Filed: Jan. 19, 2024
(86) PCT No.: PCT/EP2024/051242

§ 371 Date: Jul. 18, 2025

(30) Foreign Application Priority Data
Jan. 20, 2023 (EP) 23152666.6

(51) Int. Cl. A61K 47/59; C08G 81/00

(52) U.S. Cl. CPC A61K 47/59; C08G 81/00

(57) ABSTRACT

The present invention relates to the field of stabilized biopolymer compositions, particularly to stabilized biopolymer compositions comprising a liquid mixture, in particular aqueous solution, of at least one biopolymer component and at least one stabilizing surfactant. More specifically, the present invention relates to stabilized biopolymer compositions comprising an ethylene oxide/butylene oxide block copolymer capable to confer to said biopolymer composition superior properties in comparison to the presently known formulations, particularly with regard to aggregation tendency, hemolytic activity and solubility. The invention further relates to said block copolymers, to methods for preparing said stabilized compositions and to the use of said block copolymers for stabilizing an aqueous composition of said biopolymer. The invention further relates said stabilized composition for use in medicine, in particular for diagnostic and/or therapeutic applications. The invention further relates to an essentially dry biopolymer composition, comprising said biopolymer and said block copolymers and to methods for preparing said essentially dry compositions. The invention further relates to said essentially dry biopolymer composition for use in medicine, in particular for diagnostic and/or therapeutic applications. Said compositions may be formulated as pharmaceutical compositions which may be delivered via suitable routes of administration such as via oral, rectal, transmucosal, topical, ophthalmic, otologic, or intestinal administration; parenteral delivery, including intramuscular, subcutaneous, intramedullary injections, as well as intrathecal, direct intraventricular, intravenous, intraperitoneal, intranasal, or intraocular injections, as the case may be.

### FIELD OF THE INVENTION

The present invention relates to the field of stabilized biopolymer compositions, particularly to stabilized biopolymer compositions comprising a liquid mixture, in particular aqueous solution, of at least one biopolymer component and at least one stabilizing surfactant.

More specifically, the present invention relates to stabilized biopolymer compositions comprising an ethylene oxide/butylene oxide block copolymer capable to confer to said biopolymer composition superior properties in comparison to the presently known formulations, particularly with regard to aggregation tendency, hemolytic activity and solubility.

The invention further relates to said block copolymers, to methods for preparing said stabilized compositions and to the use of said block copolymers for stabilizing an aqueous composition of said biopolymer

The invention further relates said stabilized composition for use in medicine, in particular for diagnostic and/or therapeutic applications.

The invention further relates to an essentially dry biopolymer composition, comprising said biopolymer and said block copolymers and to methods for preparing said essentially dry compositions.

The invention further relates to said essentially dry biopolymer composition for use in medicine, in particular for diagnostic and/or therapeutic applications.

Said compositions may be formulated as pharmaceutical compositions which may be delivered via suitable routes of administration such as via oral, rectal, transmucosal, topical, ophthalmic, otologic, or intestinal administration; parenteral delivery, including intramuscular, subcutaneous, intramedullary injections, as well as intrathecal, direct intraventricular, intravenous, intraperitoneal, intranasal, or intraocular injections, as the case may be.

### BACKGROUND OF THE INVENTION

Due to favorable specificity and efficiency protein and cell-based therapies are finding widespread application in the treatment of a broad range of different diseases, for example cancer and autoimmune disorders. Antibodies have gained particular importance.

However, due to their complex three-dimensional structure and the presence of many functional groups, their use is hampered by their susceptibility to aggregate during storage.

Antibody, and generally speaking protein, aggregates pose a safety risk because they can potentially be immunogenic and cause severe side effects.

Prevention of aggregation and surface adsorption, as well as ensuring long-term stability of the antibody formulations, while preserving antibody tertiary structure and function is still challenging.

Currently, polysorbate 20 (POE sorbitan monolaurate, PS20) and polysorbate 80 (POE sorbitan monooleate, PS80) and Poloxamer 188 are the most frequently used surfactants in marketed biopharmaceutical formulations.

Polysorbates are highly efficient, but difficult to manage due to their complex composition. Their multi-component nature makes them difficult to describe, control and it presents a particular challenge to monitor stability and degradation products. In general, oxidative or hydrolytic degradation is common and leads to the formation of reactive impurities. The stability of drug formulations is reduced because the polysorbate concentration decreases and also because reactive degradation products with aldehyde or peroxide structures trigger chemical change or degradation of biomolecules. Free fatty acids and altered protein structures can trigger the formation of proteinaceous particles, which are known to cause immunogenic reactions and are therefore a concern to patient safety. [Dubey S, Giovannini R. Stability of Biologics and the Quest for Polysorbate Alternatives. Trends Biotechnol. 2021 June; 39 (6): 546-549. doi: 10.1016/j.tibtech.2020.10.007. Epub 2020 Oct. 30. PMID: 33139073.]

Furthermore, due to their similarity with the cell membrane lipids these surfactants may interact and disturb cellular equilibrium in erythrocytes, leading to membrane component reorganization and disturbance of the cell homeostasis which may eventually result in haemolysis. Polysorbates have also been described to cause immunogenic reactions, so a safer but equally effective stabilizer for parenteral formulations is highly desirable for the industry [Maggio, Edward. 2017. “Reducing or Eliminating Polysorbate Induced Anaphylaxis and Unwanted Immunogenicity in Biotherapeutics-Review Article.” Journal of Excipients and Food Chemicals 8 (3).]

The chemical stability of the surfactants used in the formulation is therefore a key aspect to be considered in the preparation of the specific excipient formulation of choice.

Poloxamer 188 a non-ionic surfactant with a more defined chemical structure and a better chemical stability (no ester-bonds) has been used as an alternative to polysorbates. However, poloxamer 188 is in general not as effective as a stabilizer and therefore typically requires higher concentrations to achieve a comparable performance. In many cases, the performance of poloxamer 188 cannot match that of polysorbates, for example in the presence of residual silicon oil traces in pre-filled syringes [Grapentin, C. et al.: Protein-Polydimethylsiloxane Particles in Liquid Vial Monoclonal Antibody Formulations Containing Poloxamer 188. Journal of Pharmaceutical Sciences, 2020).] I.

The stabilization challenge increases when the antibody is further engineered, coupled with other proteins or conjugated with small molecules presenting different physicochemical properties, such as small hydrophobic molecules and oligonucleotides.

An example thereof are antibody-fluorochrome conjugates as well as so-called antibody-drugs conjugates (ADCs), which are most commonly used in therapy to treat a broad range of different diseases and as detection reagents in diagnostic applications.

In that regard, the small molecule used for the labelling does influence the hydrophobicity and aggregation tendency of the antibody, which may possibly lead to cluster formation during storage and non-specific binding in imaging application.

Block-copolymers characterized by the presence of short hydrophilic blocks relative to the length of the hydrophobic block are known for their capacity to form worm-like micelles with high solubilisation capacity for poorly soluble aromatic drugs (Colloid Stability and Application in Pharmacy, edited by Tharwat F. Tadros, Weinheim 2007).

U.S. Pat. No. 5,300,295 describes the use of polyoxyalkylenes, particularly polyethylene oxide and polybutylene oxide block copolymers presenting sol-gel characteristics for use in drug delivery systems, or for ophthalmic use as a surgical aid.

WO1998/029127A1 describes the use of polyethylene oxide and polybutylene oxide block copolymers in the prevention of post-surgical adhesion formation/reformation in mammals following injury to the organs of a body cavity.

WO2003/024425A1 teaches the use of surfactants as possible surface stabilizers absorbed on the surface of nanoparticulate insulin compositions intended to facilitate insulin delivery in high dosage forms. A long list of ionic and non-ionic surfactants is described. Polyoxyalklyene block copolymers (such as polyethylene- or polybutylene oxide tri-block copolymers) are i.a. mentioned as possible surface stabilizers, however, without giving any preference to such compounds. Actually, in the experimental section preferred stabilizes selected from PVP/Sodum deoxycholic acid Pluronic F68/Sodium deoxycholic acid, and cationic surfactants designated S1001 and S1004 were reported to be absorbed on the surface of ball-milled nanoparticulate insulin and “in several instances” a smaller particle size of insulin was observed one week after milling. The surface stabilizer acts as a steric barrier to other solid insulin particles thereby preventing agglomeration and particle size growth and protects insulin from degradation. This document does not describe the preparation of insulin solutions and does not investigate such copolymers with respect to their haemolytic activity or water solubility. No stabilized biopolymer solutions are referred to therein.

U.S. Pat. No. 5,587,143 describes nanoparticles of poorly soluble diagnostic or therapeutic agents having adsorbed on their surface a triblock polymer of the Type PEO-PBuO-PEO with a molecular weight in the rage of 3.000 to 5.000 and an EO content of 60 wt.-%. Butronics B20-3000 and B20-5000 are mentioned. No biopolymer solutions are referred to therein. No stabilized biopolymer solutions are referred to therein as well. This document does not investigate such copolymers with respect to further important characteristics like their haemolytic activity or water solubility or surface tension.

EP-A-0 179 583 disclosed anhydrous compositions of poorly water so small molecule drugs admixed with a surfactant. The choice of surfactant is considered as “not critical”. Exemplified are i.a. Polysorbates 20 and 80, Pluronic 25R4 and a surfactant designated Butronic L-1 of unknown composition and molecular weight. This document does not investigate such copolymers with respect to further important characteristics like their haemolytic activity or water solubility or surface tension. WO2017/112828A1 teaches the use of block copolymers such as di- or tri block copolymers comprising polyethylene oxide units as stabilizing agents for the encapsulation of water-soluble biomolecules. Polyethylene or polybutylene oxide copolymers are presented as possible suitable candidates for the formation of the encapsulating shells.

Consequently, a first problem to be solved by the invention relates to the identification of a highly water soluble polymer that demonstrates a surface activity in the range of polysorbates, but that in contrast to the polysorbates shows a higher chemical stability (no ester bonds) and no hemolytic activity, and therefore would enable the provision of a liquid biopolymer composition, in particular, liquid antibody composition of improved long term stability, in particular in combination with a low to absent haemolytic activity.

### SUMMARY OF THE INVENTION

The above mentioned problem could, surprisingly, be solved by the provision of butronics (EO-BuO-EO block-copolymers) with a specific range of molecular weight and ethylene oxide content (EO %).

More particularly, the above mentioned problem could be solved by the provision of butronics presenting a molecular weight of more than 2 KDa and an EO % content of at least 60%.

These butronics were found to be concomitantly characterized by at least two, more particularly all the following properties:
high water solubility;
high chemical stability;
improved stabilization properties for antibody formulations regarding their capacity to inhibit antibody-surface inhibition and/or aggregation; and/or
low haemolytic activity;

Butronics with about 60 wt % EO based on the total weight of their ethylene and butylene oxide monomer units and a calculated molecular weight of 5.900 Da and higher are also able to solubilize small molecules particularly well compared to butronics of different structure and would therefore be particularly useful as stabilising agents for ADCs and antibody-based probes for imaging applications comprising such small molecules conjugated to an antibody molecule.

Butronics with about >50 to 85 wt % EO based on the total weight of their ethylene and butylene oxide monomer units and a calculated molecular weight of 3.000 Da to about 8.500 Da are particularly suitable to stabilize antibody-based formulations.

### DESCRIPTION OF DRAWINGS

FIG. 1: Illustration of selection criteria of particular exemplified butronics as regards good water solubility, low haemolytic activity and/or long term stability.

### DETAILED DESCRIPTION OF THE INVENTION

A. Abbreviations

| | |
|---|---|
| ADC | antibody drug conjugates |
| APC | antibody payload conjugates |
| API | active pharmaceutical ingredient |
| BuO or OBu | butylene oxide |
| CDCl₃ | deuterated chloroform |
| ECD | equivalent circular diameter |
| EDTA | Ethylenediaminetetraacetic acid |
| EO or OE | ethylene oxide |
| HPLC | high-performance liquid |
| | chromatography |
| ¹H-NHR | proton nuclear magnetic resonance |
| IgG: | immunoglobulin class G |
| PVDF | polyvinylidene fluoride |
| MFI | micro-flow imaging |
| PLA | poly lactic acid |
| PLGA | poly(lactic-co-glycolic acid) |
| PS20 | Polysorbate 20 |
| P188 | Poloxamer 188 |
| PS80 | Polysorbate 80 |
| PBS | phosphate-buffered saline |
| RBC | red blood cell |

### B. Definitions

### 1. General

Unless otherwise defined herein, scientific and technical terms used in connection with the present invention shall have the meanings that are commonly understood by those of ordinary skill in the art. The meaning and scope of the terms should be clear, however, in the event of any latent ambiguity, definitions provided herein take precedent over any dictionary or extrinsic definition. Further, unless otherwise required by context, singular terms shall include pluralities and plural terms shall include the singular.

In the context of the descriptions provided herein and of the appended claims, the use of “or” means “and/or” unless stated otherwise. Similarly, “comprise,” “comprises”, “comprising”, “include”, “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

The terms “about” or “approximately” indicate a potential variation of ±25% of the stated value, in particular ±15% or ±10%, more particularly ±5%, ±2% or ±1%.

The term “substantially” describes a range of values of from about 80 to 100%, such as, for example, 85-99.9%, in particular 90 to 99.9%, more particularly 95 to 99.9%, or 98 to 99.9% and especially 99 to 99.9%.

“Predominantly” refers to a proportion in the range of above 50%, as for example in the range of 51 to 100%, particularly in the range of 75 to 99.9%; more particularly 85 to 98.5%, like 95 to 99%.

If the present disclosure refers to features, parameters and ranges thereof of different degree of preference (including general, not explicitly preferred features, parameters and ranges thereof) then, unless otherwise stated, any combination of two or more of such features, parameters and ranges thereof, irrespective of their respective degree of preference, is encompassed by the disclosure of the present description.

### 2. Chemical Terms

The term “halogen” denotes in each case a fluorine, bromine, chlorine or iodine radical, in particular a fluorine radical.

“Alkyl” relates to a straight-chain or branched alkyl group having from 1 to 10, in particular 1 to 8, more particularly 1 to 4, 1 to 2 carbon atoms. Example are methyl, ethyl, n-propyl, n-butyl, n-hexyl, n-heptyl or n-octyl.

“Alkylene” relates to a straight-chain or branched hydrocarbon bridging group having from 1 to 22, or 2 to 22, 1 to 6, 3 to 6, 2 or 4, carbon atoms. Non limiting examples are —CH₂—, —(CH₂)₂—, —(CH₂)₃—, —(CH₂)₄—, —(CH₂)₁₀—, —(CH₂)₁₅—, —(CH₂)₂₀—, —(CH₂)₂₂— and the respective branched analogues thereof.

Optionally said alkylene groups may be interrupted by one or more heteroatoms, such as oxygen.

“Alkyleneoxy” relates to a radical of the formula —R—O—, wherein R is a straight-chain or branched alkylene group having from 1 to 22, or 2 to 22, 1 to 6, 3 to 6, 2 or 4, carbon atoms as defined herein.

A “polyalkylene oxide” relates to a group in which at least two, identical or different repeating units of alkyleneoxy groups as defined above are covalently linked.

“Block copolymer” defines a macromolecular entity characterized by at least two alternating structurally different polymer blocks; wherein each block consists essentially of structurally analogous, in particular identical repeating monomeric units. Within the structure of said block copolymer may optionally be present chemical moieties linking two or more alternating blocks, such as polyvalent, as for example di- or trivalent organic or inorganic moieties.

“Butronic” is to be broadly interpreted and generally refers to block copolymers essentially consisting of alternating butylene oxide bocks and ethylene oxide bocks and presenting an molar ethylene oxide content (EO %) of about 10 to less than 100 mol %, as well as a molecular weight of 1.000 and 15.000 g/mol. Particular butronics within the meaning of the invention are exemplified in the general part and the experimental section below.

“Static surface tension” within the meaning of the invention is to be preferably interpreted as the amount of energy per unit of surface area required to cause a deformation, such as a local increase, of the surface of a liquid sample presenting a given concentration of solute at the thermodynamic equilibrium at a predetermined temperature. The “static surface tension” is measured in millinewton per meter mN/m and is determined by means of the pendant drop technique.

“Ethylene oxide content” (EO %) and “butylene oxide content” (BuO %) within the meaning of the invention are stated herein as either mole % (mol %) or as weight percentage (wt.-%) of ethylene oxide and butylene oxide monomer units within a given block copolymer as herein defined. If not otherwise stated “%” refers to wt.-%.

The term “EO % calculated from the atomic masses of all atoms of the copolymer molecule of formula 1 refers to a wt.-% value obtained according to the following formula:

$$EO \% = [ ( \sum masses EO ) / ( \sum masses EO + \sum masses BuO + mass Starter X ) ] * 100$$

The term “a calculated molecular weight of x to y g/mol” encompasses the integers x, y and any integer between x and y. For example a molecular weight of “2.500 to 12.500 g/mol” encompasses at least the integers:
2.500, 2.600, 2.700, 2.800, 2.900;
3.000, 3.100, 3.200, 3.300, 3.400, 3.500, 3.600, 3.700, 3.800, 3.900;
4.000, 4.100, 4.200, 4.300, 4.400, 4.500, 4.600, 4.700, 4.800, 4.900;
5.000, 5.100, 5.200, 5.300, 5.400, 5.500, 5.600, 5.700, 5.800, 5.900;
6.000, 6.100, 6.200, 6.300, 6.400, 6.500, 6.600, 6.700, 6.800, 6.900;
7.000, 7.100, 7.200, 7.300, 7.400, 7.500, 7.600, 7.700, 7.800, 7.900;
8.000, 8.100, 8.200, 8.300, 8.400, 8.500, 8.600, 8.700, 8.800, 8.900;
9.000, 9.100, 9.200, 9.300, 9.400, 9.500, 9.600, 9.700, 9.800, 9.900;
10.000, 10.100, 10.200, 10.300, 10.400, 10.500, 10.600, 10.700, 10.800, 10.900;
11.000, 11.100, 11.200, 11.300, 11.400, 11.500, 11.600, 11.700, 11.800, 11.900;
12.000, 12.100, 12.200, 12.300, 12.400, and 12.500.

“Hemolysis” within the meaning of the invention relates to the tendency of a given excipient to cause breakdown of cells, particularly of red blood cells with consequent release of intracellular components.

“Equivalent circular diameter” (ECD) of a given, non-spherical particle, is defined as the diameter of a spherical particle which will give identical geometric, optical, electrical or aerodynamic behaviour to that of said non-spherical being examined.

Micro-flow imaging (MFI), is an analytical method, wherein microscopic images are automatically collected from a sample that passes the optics through a flow cell at a rate that is fast enough to analyze thousands of particles in a few minutes. Particle size is reported as the equivalent circular diameter (ECD), which is the diameter of a circle with the same projected area as the particle. In addition, information on particle count, shape and transparency can be obtained from the analysis software.

### 3. Biochemical Terms

The term “biopolymer” as used herein encompasses molecules, selected from oligopeptides, polypeptides, proteins, any type of antibody molecule or fragment or derivative thereof as defined below, glycosylated proteins, proteoglycans, oligo- and polynucleotides, DNA and RNA molecules, oligosaccharides, polysaccharides, as well as adducts or conjugates of such biopolymers, in particular of antibodies, with a further constituent selected from payload molecules as further defined below.

The term “antibody”, as used herein, broadly refers to any immunoglobulin (Ig) molecule comprised of four polypeptide chains, two heavy (H) chains and two light (L) chains, or any functional fragment, mutant, variant, or derivation thereof, which retains the essential epitope binding features of an Ig molecule. Such functional fragment, mutant, variant, or derivative antibody formats are known in the art. Nonlimiting embodiments of which are discussed below. A “full-length antibody”, as used herein, refers to an Ig molecule comprising four polypeptide chains, two heavy chains and two light chains. The chains are usually linked to one another via disulfide bonds. Each heavy chain is comprised of a heavy chain variable region (also referred to herein as “variable heavy chain”, or abbreviated herein as HCVR or VH) and a heavy chain constant region. The heavy chain constant region is comprised of three domains, CH1, CH2 and CH3. Each light chain is comprised of a light chain variable region (also referred to herein as “variable light chain”, or abbreviated herein as LCVR or VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. Immunoglobulin molecules can be of any type (e.g., IgG, IgE, IgM, IgD, IgA and IgY), class (e.g., IgG 1, IgG2, IgG3, IgG4, IgA1 and IgA2) or subclass.

The terms “antigen-binding portion” of an antibody (or simply “antibody portion”), “antigen-binding moiety” of an antibody (or simply “antibody moiety”), as used herein, refers to one or more fragments of an antibody that retain the ability to specifically bind to an antigen (i.e. the immunogenic product of the invention), i.e. are functional fragments of an antibody. It has been shown that the antigen-binding function of an antibody can be performed by one or more fragments of a full-length antibody. Such antibody embodiments may also be bispecific, dual specific, or multi-specific, specifically binding to two or more different antigens. Examples of binding fragments encompassed within the term “antigen-binding portion” of an antibody include (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., Nature 341:544-546, 1989; Winter et al., WO 90/05144 A1, herein incorporated by reference), which comprises a single variable domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al., Science 242:423-426, 1988; and Huston et al., Proc. Natl. Acad. Sci. USA 85:5879-5883, 1988). Such single chain antibodies are also encompassed within the term “antigen-binding portion” of an antibody. Other forms of single chain antibodies, such as diabodies, are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see e.g., Holliger et al., Proc. Natl. Acad. Sci. USA 90:6444-6448, 1993; Poljak et al., Structure 2:1121-1123, 1994). Such antibody binding portions are known in the art (Kontermann and Dubel eds., Antibody Engineering, Springer-Verlag. New York. 790 pp., 2001, ISBN 3-540-41354-5).

The term “antibody”, as used herein, also comprises antibody constructs. The term “antibody construct” as used herein refers to a polypeptide comprising one or more of the antigen-binding portions of the invention linked to a linker polypeptide or an immunoglobulin constant domain. Linker polypeptides comprise two or more amino acid residues joined by peptide bonds and are used to link one or more antigen binding portions. Such linker polypeptides are well known in the art (see e.g., Holliger et al., Proc. Natl. Acad. Sci. USA 90:6444-6448, 1993; Poljak et al., Structure 2:1121-1123, 1994).

An immunoglobulin constant domain refers to a heavy or light chain constant domain. Human IgG heavy chain and light chain constant domain amino acid sequences are known in the art.

Still further, a binding protein of the present invention (e.g. an antibody) may be part of a larger immunoadhesion molecule, formed by covalent or noncovalent association of the binding protein of the invention with one or more other proteins or peptides. Examples of such immunoadhesion molecules include the use of the streptavidin core region to make a tetrameric scFv molecule (Kipriyanov et al., Human Antibodies and Hybridomas 6:93-101, 1995) and use of a cysteine residue, a marker peptide and a C-terminal polyhistidine tag to make bivalent and biotinylated scFv molecules (Kipriyanov et al., Mol. Immunol. 31:1047-1058, 1994). Antibody portions, such as Fab and F(ab′) 2 fragments, can be prepared from whole antibodies using conventional techniques, such as papain or pepsin digestion, respectively, of whole antibodies. Moreover, antibodies, antibody portions and immunoadhesion molecules can be obtained using standard recombinant DNA techniques, as described herein.

An “isolated antibody”, as used herein, is intended to refer to an antibody that is substantially free of other antibodies having different antigenic specificities. An isolated antibody that specifically binds the immunogenic product of the invention may, however, have cross-reactivity to other antigens, such as Aβ globulomers, e.g. Aβ(20-42) globulomer or other Aβ forms. Moreover, an isolated antibody may be substantially free of other cellular material and/or chemicals and/or any other targeted Aβ form.

The term “human antibody”, as used herein, is intended to include antibodies having variable and constant regions derived from human germline immunoglobulin sequences. The human antibodies of the invention may include amino acid residues not encoded by human germline immunoglobulin sequences (e.g. mutations introduced by random or site-specific mutagenesis in vitro or by somatic mutation in vivo), for example in the CDRs and in particular in CDR3. However, the term “human antibody”, as used herein, is not intended to include antibodies in which CDR sequences derived from the germline of another mammalian species, such as a mouse, have been grafted onto human framework sequences.

The term “recombinant human antibody”, as used herein, is intended to include all human antibodies that are prepared, expressed, created or isolated by recombinant means, such as antibodies expressed using a recombinant expression vector transfected into a host cell (described further in Section B, below), antibodies isolated from a recombinant, combinatorial human antibody library (Hoogenboom, TIB Tech. 15:62-70, 1997; Azzazy and Highsmith, Clin. Biochem. 35:425-445, 2002; Gavilondo J. V., and Larrick J. W. (2002) BioTechniques 29:128-145; Hoogenboom H., and Chames P. (2000) Immunology Today 21:371-378), antibodies isolated from an animal (e.g. a mouse) that is transgenic for human immunoglobulin genes (see e.g. Taylor, L. D., et al. (1992) Nucl. Acids Res. 20:6287-6295; Kellermann S-A., and Green L. L. (2002) Current Opinion in Biotechnology 13:593-597; Little M. et al (2000) Immunology Today 21:364-370) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

The term “chimeric antibody” refers to antibodies which comprise heavy and light chain variable region sequences from one species and constant region sequences from another species, such as antibodies having murine heavy and light chain variable regions linked to human constant regions.

The term “CDR-grafted antibody” refers to antibodies which comprise heavy and light chain variable region sequences from one species but in which the sequences of one or more of the CDR regions of VH and/or VL are replaced with CDR sequences of another species, such as antibodies having murine CDRs (e.g., CDR3) in which one or more of the murine variable heavy and light chain regions has been replaced with human variable heavy and light chain sequences.

The terms “Kabat numbering”, “Kabat definitions and “Kabat labeling” are used interchangeably herein. These terms, which are recognized in the art, refer to a system of numbering amino acid residues which are more variable (i.e. hypervariable) than other amino acid residues in the heavy and light chain variable regions of an antibody, or an antigen binding portion thereof (Kabat et al. (1971) Ann. NY Acad, Sci. 190:382-391 and, Kabat, E. A., et al. (1991) Sequences of Proteins of Immunological Interest, Fifth Edition, U.S. Department of Health and Human Services, NIH Publication No. 91-3242). For the heavy chain variable region, the hypervariable region ranges from amino acid positions 31 to 35 for CDR1, amino acid positions 50 to 65 for CDR2, and amino acid positions 95 to 102 for CDR3. For the light chain variable region, the hypervariable region ranges from amino acid positions 24 to 34 for CDR1, amino acid positions 50 to 56 for CDR2, and amino acid positions 89 to 97 for CDR3.

As used herein, the terms “acceptor” and “acceptor antibody” refer to the antibody or nucleic acid sequence providing or encoding at least 80%, at least 85%, at least 90%, at least 95%, at least 98% or 100% of the amino acid sequences of one or more of the framework regions. In some embodiments, the term “acceptor” refers to the antibody amino acid or nucleic acid sequence providing or encoding the constant region(s). In yet another embodiment, the term “acceptor” refers to the antibody amino acid or nucleic acid sequence providing or encoding one or more of the framework regions and the constant region(s). In a specific embodiment, the term “acceptor” refers to a human antibody amino acid or nucleic acid sequence that provides or encodes at least 80%, for example at least 85%, at least 90%, at least 95%, at least 98%, or 100% of the amino acid sequences of one or more of the framework regions. In accordance with this embodiment, an acceptor may contain at least 1, at least 2, at least 3, least 4, at least 5, or at least 10 amino acid residues that does (do) not occur at one or more specific positions of a human antibody. An acceptor framework region and/or acceptor constant region(s) may be, e.g., derived or obtained from a germline antibody gene, a mature antibody gene, a functional antibody (e.g., antibodies well-known in the art, antibodies in development, or antibodies commercially available).

As used herein, the term “CDR” refers to the complementarity determining region within antibody variable sequences. There are three CDRs in each of the variable regions of the heavy chain and the light chain, which are designated CDR1, CDR2 and CDR3, for each of the variable regions. The term “CDR set” as used herein refers to a group of three CDRs that occur in a single variable region capable of binding the antigen. The exact boundaries of these CDRs have been defined differently according to different systems. The system described by Kabat (Kabat et al., Sequences of Proteins of Immunological Interest (National Institutes of Health, Bethesda, Md. (1987) and (1991)) not only provides an unambiguous residue numbering system applicable to any variable region of an antibody, but also provides precise residue boundaries defining the three CDRs. These CDRs may be referred to as Kabat CDRs. Chothia and coworkers (Chothia & Lesk, J. Mol. Biol. 196:901-917 (1987) and Chothia et al., Nature 342:877-883 (1989)) found that certain sub-portions within Kabat CDRs adopt nearly identical peptide backbone conformations, despite having great diversity at the level of amino acid sequence. These sub-portions were designated as L1, L2 and L3 or H1, H2 and H3 where the “L” and the “H” designates the light chain and the heavy chains regions, respectively. These regions may be referred to as Chothia CDRs, which have boundaries that overlap with Kabat CDRs. Other boundaries defining CDRs overlapping with the Kabat CDRs have been described by Padlan (FASEB J. 9:133-139 (1995)) and MacCallum (J Mol Biol 262 (5): 732-45 (1996)). Still other CDR boundary definitions may not strictly follow one of the above systems, but will nonetheless overlap with the Kabat CDRs, although they may be shortened or lengthened in light of prediction or experimental findings that particular residues or groups of residues or even entire CDRs do not significantly impact antigen binding. The methods used herein may utilize CDRs defined according to any of these systems, particular embodiments use Kabat or Chothia defined CDRs.

As used herein, the term “canonical” residue refers to a residue in a CDR or framework that defines a particular canonical CDR structure as defined by Chothia et al. (J. Mol. Biol. 196:901-907 (1987); Chothia et al., J. Mol. Biol. 227:799 (1992), both are incorporated herein by reference). According to Chothia et al., critical portions of the CDRs of many antibodies have nearly identical peptide backbone confirmations despite great diversity at the level of amino acid sequence. Each canonical structure specifies primarily a set of peptide backbone torsion angles for a contiguous segment of amino acid residues forming a loop.

As used herein, the terms “donor” and “donor antibody” refer to an antibody providing one or more CDRs. In one embodiment, the donor antibody is an antibody from a species different from the antibody from which the framework regions are obtained or derived. In the context of a humanized antibody, the term “donor antibody” refers to a non-human antibody providing one or more CDRs.

As used herein, the term “framework” or “framework sequence” refers to the remaining sequences of a variable region minus the CDRs. Because the exact definition of a CDR sequence can be determined by different systems, the meaning of a framework sequence is subject to correspondingly different interpretations. The six CDRs (CDR-L1, -L2, and -L3 of light chain and CDR-H1, —H2, and —H3 of heavy chain) also divide the framework regions on the light chain and the heavy chain into four sub-regions (FR1, FR2, FR3 and FR4) on each chain, in which CDR1 is positioned between FR1 and FR2, CDR2 between FR2 and FR3, and CDR3 between FR3 and FR4. Without specifying the particular sub-regions as FR1, FR2, FR3 or FR4, a framework region, as referred by others, represents the combined FR's within the variable region of a single, naturally occurring immunoglobulin chain. As used herein, a FR represents one of the four sub-regions, and FRs represents two or more of the four sub-regions constituting a framework region.

Human heavy chain and light chain acceptor sequences are known in the art.

As used herein, the term “germline antibody gene” or “gene fragment” refers to an immunoglobulin sequence encoded by non-lymphoid cells that have not undergone the maturation process that leads to genetic rearrangement and mutation for expression of a particular immunoglobulin. (See, e.g., Shapiro et al., Crit. Rev. Immunol. 22 (3): 183-200 (2002); Marchalonis et al., Adv Exp Med Biol. 484:13-30 (2001)). One of the advantages provided by various embodiments of the present invention stems from the recognition that germline antibody genes are more likely than mature antibody genes to conserve essential amino acid sequence structures characteristic of individuals in the species, hence less likely to be recognized as from a foreign source when used therapeutically in that species.

As used herein, the term “key residues” refer to certain residues within the variable region that have more impact on the binding specificity and/or affinity of an antibody, in particular a humanized antibody. A key residue includes, but is not limited to, one or more of the following: a residue that is adjacent to a CDR, a potential glycosylation site (can be either N- or O-glycosylation site), a rare residue, a residue capable of interacting with the antigen, a residue capable of interacting with a CDR, a canonical residue, a contact residue between heavy chain variable region and light chain variable region, a residue within the Vernier zone, and a residue in the region that overlaps between the Chothia definition of a variable heavy chain CDR1 and the Kabat definition of the first heavy chain framework.

As used herein, the term “humanized antibody” is an antibody or a variant, derivative, analog or portion thereof which immunospecifically binds to an antigen of interest and which comprises a framework (FR) region having substantially the amino acid sequence of a human antibody and a complementary determining region (CDR) having substantially the amino acid sequence of a non-human antibody. As used herein, the term “substantially” in the context of a CDR refers to a CDR having an amino acid sequence at least 90%, at least 95%, at least 98% or at least 99% identical to the amino acid sequence of a non-human antibody CDR. A humanized antibody comprises substantially all of at least one, and typically two, variable domains (Fab, Fab′, F(ab′) 2, FabC, Fv) in which all or substantially all of the CDR regions correspond to those of a non-human immunoglobulin (i.e., donor antibody) and all or substantially all of the framework regions are those of a human immunoglobulin consensus sequence. According to one aspect, a humanized antibody also comprises at least a portion of an immunoglobulin constant region (Fc), typically that of a human immunoglobulin. In some embodiments, a humanized antibody contains both the light chain as well as at least the variable domain of a heavy chain. The antibody also may include the CH1, hinge, CH2, CH3, and CH4 regions of the heavy chain. In some embodiments, a humanized antibody only contains a humanized light chain. In some embodiments, a humanized antibody only contains a humanized heavy chain. In specific embodiments, a humanized antibody only contains a humanized variable domain of a light chain and/or of a heavy chain.

The humanized antibody can be selected from any class of immunoglobulins, including IgM, IgG, IgD, IgA and IgE, and any isotype, including without limitation IgG 1, IgG2, IgG3 and IgG4. The humanized antibody may comprise sequences from more than one class or isotype, and particular constant domains may be selected to optimize desired effector functions using techniques well-known in the art.

The framework and CDR regions of a humanized antibody need not correspond precisely to the parental sequences, e.g., the donor antibody CDR or the consensus framework may be mutagenized by substitution, insertion and/or deletion of at least one amino acid residue so that the CDR or framework residue at that site does not correspond to either the donor antibody or the consensus framework. In one embodiment, such mutations, however, will not be extensive. Usually, at least 90%, at least 95%, at least 98%, or at least 99% of the humanized antibody residues will correspond to those of the parental FR and CDR sequences. As used herein, the term “consensus framework” refers to the framework region in the consensus immunoglobulin sequence. As used herein, the term “consensus immunoglobulin sequence” refers to the sequence formed from the most frequently occurring amino acids (or nucleotides) in a family of related immunoglobulin sequences (See e.g., Winnaker, From Genes to Clones (Verlagsgesellschaft, Weinheim, Germany 1987)). In a family of immunoglobulins, each position in the consensus sequence is occupied by the amino acid occurring most frequently at that position in the family. If two amino acids occur equally frequently, either can be included in the consensus sequence.

As used herein, “Vernier” zone refers to a subset of framework residues that may adjust CDR structure and fine-tune the fit to antigen as described by Foote and Winter (1992, J. Mol. Biol. 224:487-499, which is incorporated herein by reference). Vernier zone residues form a layer underlying the CDRs and may impact on the structure of CDRs and the affinity of the antibody.

The term “antibody”, as used herein, also comprises multivalent binding proteins. The term “multivalent binding protein” is used in this specification to denote a binding protein comprising two or more antigen binding sites. The multivalent binding protein is engineered to have the three or more antigen binding sites, and is generally not a naturally occurring antibody. The term “multispecific binding protein” refers to a binding protein capable of binding two or more related or unrelated targets. Dual variable domain (DVD) binding proteins as used herein, are binding proteins that comprise two or more antigen binding sites and are tetravalent or multivalent binding proteins. Such DVDs may be monospecific, i.e. capable of binding one antigen or multispecific, i.e. capable of binding two or more antigens. DVD binding proteins comprising two heavy chain DVD polypeptides and two light chain DVD polypeptides are referred to a DVD Ig. Each half of a DVD Ig comprises a heavy chain DVD polypeptide, and a light chain DVD polypeptide, and two antigen binding sites. Each binding site comprises a heavy chain variable domain and a light chain variable domain with a total of 6 CDRs involved in antigen binding per antigen binding site. DVD binding proteins and methods of making DVD binding proteins are disclosed in U.S. patent application Ser. No. 11/507,050 and incorporated herein by reference.

The term “labeled binding protein”, as used herein, refers to a binding protein with a label incorporated that provides for the identification of the binding protein. Likewise, the term “labeled antibody” as used herein, refers to an antibody with a label incorporated that provides for the identification of the antibody. In one aspect, the label is a detectable marker, e.g., incorporation of a radiolabeled amino acid or attachment to a polypeptide of biotinyl moieties that can be detected by marked avidin (e.g., streptavidin containing a fluorescent marker or enzymatic activity that can be detected by optical or colorimetric methods). Examples of labels for polypeptides include, but are not limited to, the following: radioisotopes or radionuclides (e.g., 3H, 14C, 35S, 90Y, 99Tc, 111 In, 125I, 131I, 177Lu, 166Ho, or 153Sm); fluorescent labels (e.g., FITC, rhodamine, lanthanide phosphors), enzymatic labels (e.g., horseradish peroxidase, luciferase, alkaline phosphatase); chemiluminescent markers; biotinyl groups; predetermined polypeptide epitopes recognized by a secondary reporter (e.g., leucine zipper pair sequences, binding sites for secondary antibodies, metal binding domains, epitope tags); and magnetic agents, such as gadolinium chelates.

The term “antibody”, as used herein, also comprises antibody conjugates. The term “antibody conjugate” refers to a binding protein, such as an antibody, chemically linked to a second chemical moiety, such as a therapeutic agent.

The term “KD” (also “Kd” or “KD”), as used herein, is intended to refer to the “equilibrium dissociation constant”, and refers to the value obtained in a titration measurement at equilibrium, or by dividing the dissociation rate constant (koff) by the association rate constant (kon). The association rate constant (kon), the dissociation rate constant (koff), and the equilibrium dissociation constant (KD) are used to represent the binding affinity of a binding protein (e.g., an antibody) to an antigen. Methods for determining association and dissociation rate constants are well known in the art. Using fluorescence-based techniques offers high sensitivity and the ability to examine samples in physiological buffers at equilibrium. Other experimental approaches and instruments such as a BIAcore® (biomolecular interaction analysis) assay can be used (e.g., instrument available from BIAcore International AB, a GE Healthcare company, Uppsala, Sweden). Additionally, a KinExAR (Kinetic Exclusion Assay) assay, available from Sapidyne Instruments (Boise, Idaho) can also be used.

“Internalize” or “internalization” of an immunoglobulin molecule relates to the ability of an immunoglobulin or ADC or APC as described herein binding to a cell surface receptor to induce a receptor-mediated endocytosis upon binding.

“De-glycosylated” or “de-glycosylation” relates to the, partial and in particular complete, removal of one or more glycosyl-residues from a glycosylated species of a biomolecule, as for example a glycosylated immunoglobulin molecule. “Antibody formulation” is to be interpreted broadly and generally refers to a product in which said antibody is admixed in liquid or solid form with a pharmaceutically acceptable liquid or solid carrier comprising organic or inorganic excipients having the capacity to influence the physico-chemical properties of said antibodies.

“Antibody conjugate” refers to a binding protein, such as an antibody, chemically linked to a second chemical moiety, such as a therapeutic agent i.e. a drug, or a payload

### C. Particular Aspects and Embodiments of the Invention

The present invention relates to the following aspects and particular embodiments thereof:

A first aspect of the invention relates to a stabilized biopolymer composition, comprising a liquid mixture, particularly a solution, and even more particularly an aqueous solution of at least one biopolymer component and at least one stabilizing ethylene oxide/butylene oxide block copolymer of the general formula 1

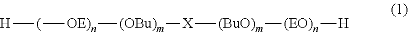

in which
X represents —O—; or a divalent organic moiety, in particular a moiety originating from an organic molecule comprising two active hydrogen atoms, such as —O—(C₂-C₄-alkylene)-O—;
m independently of each other represents an integer in the range of 4 to 25, like 10 to 22, 12 to 18 or an integer selected from 4, 5, 6, 7, 8, 9, 10, 11, 12, 13, 14, 15, 16, 17, 18, 19, 20, 21, 22, 23, 24 or 25; and
n independently of each other represents an integer in the range of 15 to 100, like 20 to 80 or 25 to 70, 30 to 60 or 35 to 65, or an integer selected from 15, 16, 17, 18, 19, 20, 21, 22, 23, 24, 25, 26, 27, 28, 29, 30; 31, 32, 33, 34, 35, 36, 37, 38, 39, 40; 41, 42, 43, 44, 45, 46, 47, 48, 49, 50; 51, 52, 53, 54, 55, 56, 57, 58, 59, 60; 61, 62, 63, 64, 65, 66, 67, 68, 69 or 70;

wherein said block copolymer shows a combination of the following features:
a) a calculated molecular weight of 2.500 to 12.500 g/mol, particularly 2.800 to 9.000 g/mol, more particularly 3.000 to 8.000 g/mol, and most particularly 3.500 to 8.000 g/mol, or 3.500 to 7.000 g/mol, in particular calculable from the sum of atomic masses of all atoms of the copolymer molecule of formula 1; and
b) an EO content of >50 to 85 wt.-%, particularly 53 to 85 wt.-%, or 55 to 80 wt.-%, more particularly 57 to 75 wt.-% or 57 to 80 wt.-%, like 60 to 70 wt.-%, each based on the dry weight of said block copolymer, in particular calculable from the atomic masses of all atoms of the copolymer molecule of formula 1.

Said ethylene oxide/butylene oxide block copolymer formally may also be designated as “triblock polymers”, as they contain a central BuO block essentially consisting of BuO monomers, flanked by two EO blocks.

As particular combinations of features a) and b) there may be mentioned
a calculated molecular weight of 2.500 to 12.500 g/mol and an EO content of >50 to 85 wt.-%,
a calculated molecular weight of 2.800 to 9.000 g/mol and an EO content of 55 to 85 wt.-%,
a calculated molecular weight of 2.800 to 9.000 g/mol and an EO content of 55 to 80 wt.-%,
a calculated molecular weight of 3.000 to 8.000 g/mol, and an EO content of 55 to 80 wt.-%,
a calculated molecular weight of 3.500 to 8.000 g/mol, an EO content of 57 to 80 wt.-%,
a calculated molecular weight of 3.000 to 8.000 g/mol and an EO content of 57 to 75 wt.-%, or
a calculated molecular weight of 3.500 to 7.000 g/mol and an EO content of 60 to 70 wt.-%,

wherein in particular, the molecular weight in each case being calculable from the sum of atomic masses of all atoms of the copolymer molecule of general formula 1 and the EO content in each case calculable from the atomic masses of all atoms of the copolymer molecule of formula 1.

Preferably, said block copolymer shows at least one of the following additional features:
c) a water solubility of at least 5 wt.-%, or more particularly at least 10% based on the total weight of the aqueous solution of the block copolymer;
d) an aqueous solution of said block copolymer (measured at a concentration of 0.1 g/l) has a surface tension SFT of less than 60 mN/m to more than 25 mN/m, in particular of 53 to 30 mN/m, like, for example, of about 35, about 40, about 45 of about 50 mN/m; and
e) a lack of hemolytic activity, in particular a hemolytic activity of less than 10% or more particularly less than 5%, or less than 1, 2, 3 or 4% hemolysis, like 0% or 0.1 to 0.9% hemolysis caused by a solution of 100 g/l block copolymer.

Each of the above features a) to e) may be determined as described below in the experimental section by applying the respective analytical method and measurement conditions.

According to a particular embodiment, a preferred subgroup of block-copolymers shows a combination of anyone of the above features a), b) and e).

According to another particular embodiment, a preferred subgroup of block-copolymers shows a combination of anyone of the above features a), b), c) and e).

According to still another particular embodiment, a preferred subgroup of block-copolymers shows a combination of anyone of the above features a), b), c), d) and e).

According to still another embodiment a biopolymer solution is provided, comprising an aqueous solution, of at least one biopolymer component and at least one ethylene oxide/butylene oxide block copolymer of the general formula 1

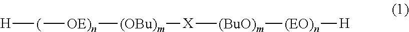

in which
X represents —O—; or a divalent organic moiety
m independently of each other represents an integer in the range of 4 to 25; and
n independently of each other represents an integer in the range of 15 to 100;

wherein

said biopolymer is selected from oligopeptides, polypeptides, proteins, antibody molecules or fragments or derivatives thereof, glycosylated proteins, proteoglycans, oligo- and polynucleotides, DNA and RNA molecules, oligosaccharides, polysaccharides, as well as adducts or conjugates of such biopolymers with a further constituent selected from payload molecules,

said block copolymer shows a combination of the following features:
a) a molecular weight of 2.500 to 12.500 g/mol, particularly 2.800 to 9.000 g/mol, more particularly 3.000 to 8.000 g/mol, most particularly 3.500 to 8.000 g/mol, each calculable from the sum of atomic masses of all atoms of the copolymer molecule of general formula 1;
b) an EO content of >50 to 85 wt.-%, particularly 53 to 85 wt.-%, more particularly 55 to 80 wt.-%, most particularly 57 to 80 wt.-%, each based on the dry weight of said block copolymer; calculable from the atomic masses of all atoms of the copolymer molecule of formula 1;
c) a water solubility of at least 5 wt.-% based on the total weight of the aqueous solution of the block copolymer; and
d) less than 10% hemolytic activity caused by a solution of 100 g/l, or more particularly less than 5%, or less than 1, 2, 3 or 4% hemolysis, like 0% or 0.1 to 0.9% hemolysis caused by a solution of 100 g/l block copolymer.

According to a more particular embodiment thereof, said biopolymer is selected from oligopeptides, polypeptides, proteins, antibody molecules or fragments or derivatives thereof, glycosylated proteins, as well as adducts or conjugates of such biopolymers with a further constituent selected from payload molecules

According to still another more particular embodiment thereof, said biopolymer is elected from polypeptides, proteins or glycosylated proteins, like for example enzymes,

According to still another more particular embodiment thereof, said biopolymer is selected from antibody molecules or fragments or derivatives thereof, as well as adducts or conjugates of such biopolymers with a further constituent selected from payload molecules.

According to still another more particular embodiment thereof, said biopolymer is selected from monoclonal or polyclonal antibody molecules or fragments or derivatives thereof, as well as adducts or conjugates of such biopolymers with a further constituent selected from payload molecules.

According to still another particular embodiment thereof, said biopolymer is selected from polyclonal antibodies, fragments or derivatives thereof.

According to still another particular embodiment thereof, said biopolymer is selected from monoclonal antibodies, fragments or derivatives thereof.

According to still another particular embodiment thereof, said biopolymer is selected from monoclonal antibodies, or fragments thereof, conjugated with a further constituent selected from payload molecules.

According to still another more particular embodiment thereof, said biopolymer is selected from proteoglycans, oligo- and polynucleotides, DNA and RNA molecules, oligosaccharides, and polysaccharides, as well as adducts or conjugates of such biopolymers with a further constituent selected from payload molecules.

In another particular embodiment of said first aspect, a stabilized biopolymer composition is provided, wherein said butylene oxide block is composed of monomer units derived from 1,2-butylene oxide, 2,3-butylene oxide, isobutylene oxide, or mixtures thereof, in particular essentially form 1,2-butylene oxide, and more particularly from 1,2-butylene oxide.

In another particular embodiment of said first aspect, a stabilized biopolymer composition is provided, wherein X is selected from —O—; —O-alkylene-O—, in particular-O—(C₂-C₂₂-alkylene)-O—, more particularly —O—(C₃-C₆-alkylene)-O—, wherein the alkylene chain is straight-chained or branched, and is optionally interrupted by one or more heteroatoms, in particular oxygen atoms; more particularly —O—, —O-(n-butylene)-O—, —O-(1,2-butylene)-O—, a group of formula 2 below, like especially —O-(n-butylene)-O—; or X is a group of the formula 2

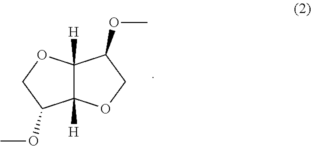

According to another particular embodiment of said first aspect, the invention relates to a stabilized biopolymer composition, wherein said block copolymer of general formula 1 shows a combination of the following features
a) a calculated molecular weight 3.500 to 7.000 g/mole in particular calculable from the sum of atomic masses of all atoms of the copolymer molecule of formula 1, and
b) an EO content of 60 to 70 wt.-%, based on the dry weight of said block copolymer in particular calculable from the atomic masses of all atoms of the copolymer molecule of formula 1; and
c) X=—O-n-butylene-O—.

According to another particular embodiment of said first aspect, said block copolymer may be selected from the following compounds of the general formula 1, wherein X, m and n have the following meanings:
X=—O-n-butylene-O—; m=10, n=17
X=—O-n-butylene-O—; m=10, n=27
X=—O-n-butylene-O—; m=5, n=35
X=—O-n-butylene-O—; m=16, n=40
X=—O-n-butylene-O—; m=12, n=48
X=—O-n-butylene-O—; m=21, n=54
X=—O-n-butylene-O—; m=10, n=70
X=a moiety of formula 2, m=10; and n=17;
X=a moiety of formula 2, m=10, and n=27;
X=a moiety of formula 2, m=5, and n=35;
X=a moiety of formula 2, m=16, and n=40;
X=a moiety of formula 2, m=12, and n=48;
X=a moiety of formula 2, m=21, and n=54;
X=a moiety of formula 2, m=11, and n=68; or
X=a moiety of formula 2, m=10, and n=70.

According to still another more particular embodiment a biopolymer composition is provided, wherein the block copolymer is selected from the following compounds of the general formula 1, wherein X, m and n have the following meanings:
X=—O-n-butylene-O—, m=12, and n=48 and a molecular weight of approximately 6.050, calculable from the sum of atomic masses of all atoms of the copolymer molecule of general formula 1; or
X=—O-n-butylene-O—, m=10, and n=70 and a molecular weight of approximately 7.658, calculable from the sum of atomic masses of all atoms of the copolymer molecule of general formula 1;
X=—O-n-butylene-O—, m=16, and n=40 and a molecular weight of approximately 5.922, calculable from the sum of atomic masses of all atoms of the copolymer molecule of general formula 1; or
X=a moiety of formula 2, m=11, and n=68 and a molecular weight of approximately 7.724, calculable from the sum of atomic masses of all atoms of the copolymer molecule of general formula 1.

According to still another more particular embodiment a biopolymer composition is provided, wherein X is a group of the formula 2

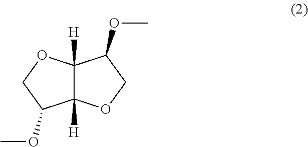

and wherein
said block copolymer shows a combination of the following features
   a) a molecular weight of 3.000 to 8.000 g/mol, 5.500 to 8.000 g/mol, and especially 5.800 to 7.900 g/mol, in particular calculable from the sum of atomic masses of all atoms of the copolymer molecule of general formula 1;
   b) an EO content of 55 to 85 wt.-% based on the dry weight of said block copolymer; in particular calculable from the atomic masses of all atoms of the copolymer molecule of formula 1;
   c) a water solubility of at least 5 wt.-% based on the total weight of the aqueous solution of the block copolymer; and
   d) less than 10% hemolytic activity caused by a solution of 100 g/l.

According to still another particular embodiment said biopolymer is an immunoglobulin or protein molecule or an antibody payload conjugate (APC), particularly an antibody drug conjugate (ADC), each optionally glycosylated, wherein said at least one ethylene oxide/butylene oxide block copolymer is of the above general formula 1,
in which
X represents —O—; or a divalent organic moiety;
m independently of each other represents an integer in the range of 10 to 20; and
n independently of each other represents an integer in the range of 25 to 75;

wherein said block copolymer shows a combination of the following features:
a) a molecular weight of 3.500 to 8.000 g/mol, in particular calculable from the sum of atomic masses of all atoms of the copolymer molecule of general formula 1 and
b) an EO content of particularly 55 to 85 wt.-%, based on the dry weight of said block copolymer, in particular calculable from the atomic masses of all atoms of the copolymer molecule of formula 1;
c) a water solubility of at least 5 wt.-% based on the total weight of the aqueous solution of the block copolymer; and
d) less than 10% hemolytic activity caused by a solution of 100 g/l.

According to another particular embodiment of said first aspect, the invention relates to a stabilized biopolymer composition, wherein the biopolymer is selected from oligopeptides, polypeptides, proteins, glycosylated proteins, proteoglycans, antibody molecules or fragments or derivatives thereof, adducts or conjugates of such biopolymers with a further constituent selected from payload molecules, in particular
a) pharmaceutically active compounds;
b) labeling agents;
c) biological small molecules such as lipids, phospholipids, glycolipids, sterols, vitamins, hormones, neurotransmitters, amino acids, nucleotides, monosaccharides; or
d) biological macromolecules such as peptides, oligopeptides, polypeptides, proteins, nucleic acids, such as any forms of DNA and RNA, oligosaccharides, and polysaccharides.

In the latter embodiment, said biopolymer may preferably be a diagnostically applicable or a therapeutically active biopolymer.

In the latter two embodiments, said biopolymer may preferably be selected from proteins, in particular enzymes and immunoglobulin molecules, each optionally glycosylated.

In the latter three embodiments said biopolymer may preferably be selected from adducts or conjugates of an immunoglobulin molecule and a payload molecule.

In the latter embodiment, said biopolymer may preferably be an antibody payload conjugate (APC), particularly antibody drug conjugate (ADC).

In a particular embodiment of said first aspect, a stabilized biopolymer composition is provided, wherein said block copolymer of the general formula (I) is contained in a proportion of 0.001 to 30% or 0.001 to 10%, like 0.01 to 8%, 0.1 to 5% or 1 to 3%, each based on the total weight of the liquid composition.

In a further embodiment of said first aspect, a stabilized biopolymer composition is provided, in which said biopolymer is contained in a proportion of 0.01 to 30%, like 0.1 to 25%, 1 to 20%, 3 to 15% or 5 to 10%, each based on the total weight of the liquid composition.

In a particular embodiment of said first aspect, a stabilized biopolymer composition is provided, which is optionally in buffered form, having a pH in the range of 5 to 9, particularly 6, 7 or 8.

According to a second aspect, the invention relates to an essentially dry biopolymer composition, comprising at least one biopolymer component as defined in the above-identified first aspect and at least one stabilizing ethylene oxide/butylene oxide block copolymer of the general formula (1) as defined in the above-identified first aspect.

In preferred embodiments said essentially dry biopolymer composition has a liquid content of 0% to 5% wt %, as for example 0.1 to 4.5 wt %, like 1, 2, 3 or 4 wt %, based on the total weight of said composition.

Optionally in said essentially dry biopolymer composition said at least one block copolymer of the general formula (1) (=A) and said at least one biopolymer (=B) are contained in a weight ratio (A):(B) in the range of 1:20.000 to 10:1, or 1:5.000 to 2:1 or of 1:100 to 1,2:1, particularly 1:50 to 5:1, or 1:20 to 2:1, and more particularly 1:10 to 1,1:1.

In preferred embodiments said essentially dry biopolymer composition is characterized in that said block copolymer and said at least one biopolymer together are contained in a proportion of 1 to less than 100 wt %, in particular 5 to 60 wt %, more particular 10 to 50 wt %, or 20 to 40 wt. % or even 20 to 25% based on the total weight of said essentially dry composition.

Optionally said essentially dry biopolymer composition comprises at least one further excipient in a proportion of 0.1 to 99%, 40 to 95% and 50 to 90% wt.-% based on the total dry weight of said essentially dry composition.

According to another particular embodiment a liquid or essentially dry biopolymer composition is provided, comprising a nanoconstruct, in particular nanoparticles, composed of at least one biopolymer component as defined above and at least one stabilizing ethylene oxide/butylene oxide block copolymer of the general formula (1) as defined above.

More particularly, said nanoconstruct, or nanoparticle is composed of at least one biodegradable polymer (e.g. PLA, PLGA) or at least one lipid in addition to the at least one stabilizing ethylene oxide/butylene oxide block copolymer of the general formula (1) as defined above.

Even more particularly, said at least one biopolymer component is DNA, RNA or peptides.

A third aspect the invention relates to the use of the block copolymer defined in the above-identified first aspect for stabilizing an aqueous composition, in particular an aqueous solution, of a least one biopolymer as defined in the above-identified first aspect.

A fourth aspect the invention relates to the composition according to the above-identified first or second aspect for use in medicine, in particular for diagnostic and/or therapeutic applications.

According to a further embodiment the invention relates to a composition according to the above-identified first or second aspect, which is a pharmaceutical composition optionally further supplemented by at least one pharmaceutically acceptable excipient.

A fifth aspect the invention relates to a method of preparing a stabilized composition according to the above-identified first aspect, which method comprises
a) preparing in any order an aqueous, optionally buffered solution of the biopolymer; and an aqueous, optionally buffered solution of the block copolymer of general formula (I) and
b) preparing a mixture of both aqueous solutions as obtained in step a).

A sixth aspect the invention relates to a method of preparing the essentially dry stabilized composition according to the second aspect, which method comprises
a) preparing in any order an aqueous, optionally buffered solution of said biopolymer; and an aqueous, optionally buffered solution of said block copolymer of general formula (1) and
b) preparing a mixture of both aqueous, optionally buffered, solutions as obtained in step a);
c) optionally supplementing the aqueous, optionally buffered solutions prepared in step a) and/or the mixture of both aqueous, optionally buffered, solutions prepared in step b), with at least one pharmaceutically acceptable excipient;
d) drying the mixture obtained in step b) or c).

Said drying step d) is performed by conventional, well-known methods. For example the drying step d) is performed by spray-drying or freeze-drying the mixture obtained in step b) or c).

A seventh aspect the invention relates to a block copolymer selected from the following compounds of the general formula (I), wherein X, m and n have the following meanings:
X=—O-n-butylene-O—, m=10; and n=17;
X=—O-n-butylene-O—, m=10, and n=27;
X=—O-n-butylene-O—, m=5, and n=35;
X=—O-n-butylene-O—, m=16, and n=40;
X=—O-n-butylene-O—, m=12, and n=48;
X=—O-n-butylene-O—, m=21, and n=54;
X=—O-n-butylene-O—, m=10, and n=70;
X=a moiety of formula 2, m=10; and n=17;
X=a moiety of formula 2, m=10, and n=27;
X=a moiety of formula 2, m=5, and n=35;
X=a moiety of formula 2, m=16, and n=40;
X=a moiety of formula 2, m=12, and n=48;
X=a moiety of formula 2, m=21, and n=54;
X=a moiety of formula 2, m=11, and n=68; or
X=a moiety of formula 2, m=10, and n=70.

In particular, the invention relates to a block copolymer selected from the following compounds of the general formula (I), wherein X, m and n have the following meanings:
X=—O-n-butylene-O—, m=12, and n=48 and a molecular weight of approximately 6.050, calculable from the sum of atomic masses of all atoms of the copolymer molecule of general formula 1;
X=—O-n-butylene-O—, m=10, and n=70 and a molecular weight of approximately 7.658, calculable from the sum of atomic masses of all atoms of the copolymer molecule of general formula 1;
X=—O-n-butylene-O—, m=16, and n=40 and a molecular weight of approximately 5.922, calculable from the sum of atomic masses of all atoms of the copolymer molecule of general formula 1; or
X=a moiety of formula 2, m=11, and n=68 and a molecular weight of approximately 7.724, calculable from the sum of atomic masses of all atoms of the copolymer molecule of general formula 1.

### D. Further Embodiments

### D.1. Synthesis of Block Copolymers of Formula (1)

Methods for the preparation of stabilizing ethylene oxide/butylene oxide block copolymers of the general formula 1 are generally known in the art.

Suitable methods for their preparation are reported, for example, in U.S. Pat. No. 2,828,345.

Briefly, said block copolymers may, for example be prepared by a multistep protocol which foresees a first step in which an organic molecule comprising two active hydrogen atoms, like 1,4-butane diol or 1,3-butane diol, and butylene oxide are condensed to form a polyoxybutylene. Thereafter, ethylene oxide is added and the reaction is let to proceed until the desired oxyethylene content is reached.

Examples of suitable butylene oxides are 1,2-butylene oxide and 2,3-butylene oxide.

The reaction is preferably carried out under moisture-free conditions at elevate temperature and in presence of a suitable catalyst such as an alkali metal hydroxide or alkoxide, like alkali metal tert. butoxide.

The reaction can be carried out in presence of water, whereas reacting butylene oxide with water (in particular originating from an aqueous catalyst solution, or water contained in the starter or EO or BuO as added to the reaction (as further detailed in the experimental section below) forms in situ an organic molecule with two active hydrogen groups, e.g. 1,2-butane diol (in analogy to the disclosure in CA 698,568).

The amount of catalyst employed should be from 0.05 to 1 percent by weight based on the total reactants. Reaction temperatures are in the range of from 80° to 200° C., with a temperature of about 110° C. or 170° C. being preferred during most of the reaction.

Superatmospheric pressures in the range of from 0.5 to 15 bar are ordinarily employed, very good results being obtained at pressures of from about 1 to 5 bar. The alkylene oxides employed are preferably substantially anhydrous, e.g. the moisture content of the oxides ordinarily should not exceed about 0.1 percent by weight. The alkylene oxides are also preferably as free as practical from contaminants, such as aldehydes, which give rise to side reactions and by-product formation.

The reaction may be conducted either batch-wise or continuously as desired. In batchwise operation, the commercially anhydrous organic molecule comprising two active hydrogen atoms, like 1,4-butane diol or butylene glycol is charged into a suitable dry reaction vessel, such as an autoclave, and mixed with an effective amount of catalyst, usually about 0.2 wt.-% of potassium hydroxide in terms of the total amount of reactants.

Prior to the introduction of butylene oxide, the reaction vessel is advantageously flushed with a stream of dry inert gas, such as nitrogen, to remove any air or oxygen therefrom. The elimination of molecular oxygen from the reaction vessel is an important factor in obtaining colorless products and may, if desired, be carried out after adding the butylene glycol and catalyst to the reaction vessel.

After these preliminaries, the mixture of -potassium hydroxide and said organic molecule comprising two active hydrogen atoms, like 1,4-butane diol or butylene glycol, is heated to a reaction temperature of about 140° C. and butylene oxide is added at a fairly rapid rate.

Usually, the rate of addition of butylene oxide is such as to maintain a pressure of about 3 bar in the reactor. Vigorous agitation is desirable to maintain a good dispersion of catalyst and uniform reaction rates throughout the mass.

The reaction of butylene oxide with butylene glycol is exothermic and it is therefore necessary to provide adequate cooling means.

By controlling the rate of addition of butylene oxide to maintain the pressure fairly constant, the reaction temperature may also be maintained constant.

The addition of butylene oxide is stopped upon obtaining the desired molecular weight of the polyoxybutylene glycol condensation product as determined by, for example, hydroxyl analysis or 1H-NMR reckoning two free hydroxyl groups per molecule. Thereafter, ethylene oxide is condensed with the polyoxybutylene glycol condensation product to give a product in accordance with the invention. The addition of ethylene oxide is carried out in the same manner as the addition of butylene oxide already described.

Purification may be conducted by heating it at a reduced pressure under reflux or by stripping with inert gas to distill off any low boiling material.

D.2. Antibody-Payload Conjugates and their Preparation

As stated above a biopolymer as part of a stabilized formulation of the invention may, in preferred embodiments, be an antibody payload conjugate (APC), and more particularly an antibody drug conjugate (ADC).

Antibody drug conjugates (ADC) combine two major therapies applied nowadays, namely chemotherapy and antibody therapy. Antibodies are important biologics, which bind to their specific antigen, e.g. to a receptor on a cell, which is overexpressed on a diseased cell, like a cancer cell compared to a healthy cell. The antibody activates the competent system and consequently, the cancer cell will be destroyed by killer cells. On the other hand, chemotherapy is the treatment with cytotoxic moieties, which can be absorbed by the cell and kill the cell by different pathways. Active cells, like cancer cells, can take up more of the cytotoxic drug than healthy cells. Nonetheless, this therapy shows huge side-effects. By combining antibodies with the killing effect of cytotoxic drugs, a directed and efficient cancer therapy is possible. Thereby, the choice of conjugation method, how the antibody is labeled with the drug, is very important.

The first ADCs on market were conjugated randomly, by utilizing cysteines or lysines of the antibody sequence to attach the toxic payload. This leads to a heterologous species with different kinds of drug-to-antibody ratios (DAR), which negatively influences pharmacokinetic and safety profile of the ADC (Senter, P. D. & Sievers, E. L. The discovery and development of brentuximab vedotin for use in relapsed Hodgkin lymphoma and systemic anaplastic large cell lymphoma. Nat. Biotechnol. 30, 631-637 (2012); Junutula, J. R. et al. Site-specific conjugation of a cytotoxic drug to an antibody improves the therapeutic index. Nat. Biotechnol. 26, 925-932 (2008)).

Site-specific conjugation methods followed, utilizing the glycosylation of the antibody or enzymatic coupling (Van Geel, R. et al. Chemoenzymatic Conjugation of Toxic Payloads to the Globally Conserved N-Glycan of Native mAbs Provides Homogeneous and Highly Efficacious Antibody-Drug Conjugates. Bioconjug. Chem. 26, 2233-2242 (2015); Dennler, P. et al. Transglutaminase-based chemo-enzymatic conjugation approach yields homogeneous antibody-drug conjugates. Bioconjug. Chem. 25, 569-578 (2014)). These methods are restricted to specific sites and cannot be transferred to other positions in the antibody sequence.

One conjugation method, which is site-specific and unlimited in the choice of the position, is the use of the genetic code expansion technology. Therefore, a non-canonical amino acid (ncAA) is introduced at the translational level in the antibody sequence in response to a stop codon (e.g. amber stop codon, TAG), which is placed beforehand into the gene of the antibody. An orthogonal aminoacyl-tRNA-synthetase (aaRS)/tRNA pair has to be introduced into the antibody expression host, which is able to bind and introduce the ncAA into the growing antibody protein sequence (Lemke, E. A. The exploding genetic code. Chem Bio Chem 15, 1691-1694 (2014); de la Torre, D. & Chin, J. W. Reprogramming the genetic code. Nat. Rev. Genet. 22, 169-184 (2021)). The ncAA can be positioned freely in the antibody sequence and can used for the conjugation with the toxic payload depending on its chemical properties.

There are different ncAAs existing, based on various endogenous amino acids, like lysine or tryptophan. They can have different headgroups, which influences their chemical properties and give rise to which chemical reaction they can undergo. Tian et al. showed the incorporation of a ncAA containing a ketone headgroup into several antibodies expressed in CHO cells followed by coupling to a cytotoxic payload via copper-free click reaction. The reaction between an alkoxyamine functional group and the ketone could only be done at pH4, otherwise requiring additives (Tian, F. et al. A general approach to site-specific antibody drug conjugates. Proc. Natl. Acad. Sci. U.S.A. 111, 1766-1771 (2014)).

The fastest bio-orthogonal chemical reaction nowadays, which can be even done at neutral pH, is the strain-promoted inverse electron demand Diels-Alder cycloaddition (SPIEDAC) between strained alkene or alkyne and a tetrazine group (Nikić, I. & Lemke, E. A. Genetic code expansion enabled site-specific dual-color protein labeling: superresolution microscopy and beyond. Curr. Opin. Chem. Biol. 28, 164-173 (2015)).

One special case of a SPIEDAC reaction is the conjugation of a cyclooctene-lysine (SCO) and a 1,2,4,5-tetrazine, which might not be an inverse electron demand reaction and does not show the same reaction speed as other strained alkenes or alkynes. Therefore, SCO as well as the resulting reaction product, shows highest stability in the cellular environment compared to other strained alkene/alkynes tested (Wagner, J. A., Mercadante, D., Nikic, I., Lemke, E. A. & Gräter, F. Origin of Orthogonality of Strain-Promoted Click Reactions. Chem.—A Eur. J. 21, 12431-12435 (2015); Reinkemeier, C. D. et al. Synthesis and Evaluation of Novel Ring-Strained Noncanonical Amino Acids for Residue-Specific Bioorthogonal Reactions in Living Cells. Chem.—A Eur. J. 27, chem.202100322 (2021)).

The toxic payload can be divided into a linker and a cytotoxic drug. There are many linker technologies existing nowadays, ranging from non-cleavable, to enzymatic, acidic and glutathione cleavable linkers. The linker is directly influencing the pharmacokinetics and pharmacodynamics of the ADC (Hafeez, U., Parakh, S., Gan, H. K. & Scott, A. M. Antibody-drug conjugates for cancer therapy. Molecules 25, 4764 (2020); Khongorzul, P., Ling, C. J., Khan, F. U., Ihsan, A. U. & Zhang, J. Antibody-Drug Conjugates: A Comprehensive Review. Mol. Cancer Res. 18, 3-19 (2020)).

A handful of different cytotoxic drug families are used nowadays as chemical warhead for an ADC, like auristatins, maytansinoids, calicheamicins and duocarmycins. They are either damaging DNA or microtubuli (Chau, C. H., Steeg, P. S. & Figg, W. D. Antibody-drug conjugates for cancer. Lancet 394, 793-804 (2019); Sievers, E. L. & Senter, P. D. Antibody-drug conjugates in cancer therapy. Annu. Rev. Med. 64, 15-29 (2013)).

Non-limiting examples of suitable antibodies and payload molecules within the meanings of the present invention are reported in the sections below.

### D.3. Antibodies

The term “antibody” within the meaning of the present invention designates equally antibodies, antibody derivatives, antibody fragments, antibody (fragment) fusions (e.g. bi-specific and tri-specific mAb fragments or derivatives), polyclonal or monoclonal antibodies, such as human, humanized, mouse or chimeric antibodies (see also general definition provided above).

Typical non-limiting examples are selected form biologically, in particular pharmacologically active antibody molecules.

Non-limiting examples are selected form the following group: trastuzumab, bevacizumab, cetuximab, panitumumab, ipilimumab, rituximab, alemtuzumab, ofatumumab, gemtuzumab, brentuximab, ibritumomab, tositumomab, pertuzumab, adecatumumab, IGN101, INA01 labetuzumab, hua33, pemtumomab, oregovomab, minretumomab (CC49), cG250, J591, MOv-18, farletuzumab (MORAb-003), 3F8, ch14, 18, KW-2871, hu3S193, IgN31 1, IM-2C6, CDP-791, etaracizumab, volociximab, nimotuzumab, MM-121, AMG 102, METMAB, SCH 900105, AVE1642, IMC-A12, MK-0646, R1507, CP 751871, KB004, III A4, mapatumumab, HGS-ETR2, CS-1008, denosumab, sibrotuzumab, F19, 81 C6, pinatuzumab, lifastuzumab, glembatumumab, coltuximab, lorvotuzumab, indatuximab, anti-PSMA, MLN-0264, ABT-414, milatuzumab, ramucirumab, abagovomab, abituzumab, adecatumumab, afutuzumab, altumomab pentetate, amatuximab, anatumomab, anetumab, apolizumab, arcitumomab, ascrinvacumab, atezolizumab, bavituximab, bectumomab, belimumab, bivatuzumab, brontictuzumab, cantuzumab, capromab, catumaxomab, citatuzumab, cixutumumab, clivatuzumab, codrituzumab, conatumumab, dacetuzumab, dallotuzumab, daratumumab, demcizumab, denintuzumab, depatuxizumab, derlotuximab, detumomab, dinutuximab, drozitumab, duligotumab, durvalumab, dusigitumab, ecromeximab, edrecolomab, elgemtumab, emactuzumab, enavatuzumab emibetuzumab, enfortumab, enoblituzumab, ensituximab, epratuzumab, ertumaxomab, etaracizumab, farletuzumab, ficlatuzumab, figitumumab, flanvotumab, futuximab, galiximab, ganitumab, icrucumab, igovomab, imalumab, imgatuzumab, indusatumab, inebilizumab, intetumumab, iratumumab, isatuximab, lexatuzumab, lilotomab, lintuzumab, lirilumab, lucatumumab, lumretuzumab, margetuximab, matuzumab, mirvetuximab, mitumomab, mogamulizumab, moxetumomab, nacolomab, naptumomab, narnatumab, necitumumab, nesvacumab, nimotuzumab, nivolumab, nofetumomab, obinutuzumab, ocaratuzumab, ofatumumab, olaratumab, onartuzumab, ontuxizumab, oportuzumab, oregovomab, otlertuzumab, pankomab, parsatuzumab, pasotuxizumab, patritumab, pembrolizumab, pemtumomab, pidilizumab, pintumomab, polatuzumab, pritumumab, quilizumab, racotumomab, ramucirumab, rilotumumab, robatumumab, sacituzumab, samalizumab, satumomab, seribantumab, siltuximab, sofituzumab, tacatuzumab, taplitumomab, tarextumab, tenatumomab, teprotumumab, tetulomab, ticilimumab, tigatuzumab, tositumomab, tovetumab, tremelimumab, tucotuzumab, ublituximab, ulocuplumab, urelumab, utomilumab, vadastuximab, vandortuzumab, vantictumab, vanucizumab, varlilumab, veltuzumab, vesencumab, volociximab, vorsetuzumab votumumab, zalutumumab, zatuxima, combination and derivatives thereof, as well as other monoclonal antibodies targeting CAI 25, CAI 5-3, CAI 9-9, L6, Lewis Y, Lewis X, alpha fetoprotein, CA 242, placental alkaline phosphatase, prostate specific antigen, prostate specific membrane antigen, prostatic acid phosphatase, epidermal growth factor, MAGE-1, MAGE-2, MAGE-3, MAGE-4, transferrin receptor, p97, MUCI, CEA, gplOO, MARTI, IL-2 receptor, CD20, CD52, CD33, CD22, human chorionic gonadotropin, CD38, CD40, mucin, P21, MPG, and Neu oncogene product.

### D.4. Payload Molecules

Payload molecules typically used may be selected from bioactive compounds, in particular drugs, labeling agents, and chelators. Non-limiting examples thereof are given in the following sections.

### D.4.1. Bioactive Compounds

Bioactive compounds include, but are not limited to, the following:

Bioactive compounds applicable according to the present invention include but are not limited to: small organic molecule drugs, steroids, lipids, proteins, aptamers, oligopeptides, oligonucleotides, oligosaccharides, as well as peptides, peptoids, amino acids, nucleotides, oligo- or polynucleotides, nucleosides, DNA, RNA, toxins, glycans and immunoglobulins.

Exemplary classes of bioactive compounds that can be used in the practice of the present invention include but are not limited to hormones, cytotoxins, antiproliferative/antitumor agents, antiviral agents, antibiotics, cytokines, anti-inflammatory agents, antihypertensive agents, chemosensitizing, photosensitizing and radiosensitizing agents, anti-AIDS substances, anti-viral agents, immunosuppressants, immunostimulants, enzyme inhibitors, anti-Parkinson agents, neurotoxins, channel blockers, modulators of cell-extracellular matrix interactions including cell growth inhibitors and anti-adhesion molecules, inhibitors of DNA, RNA or protein synthesis, steroidal and non-steroidal anti-inflammatory agents, anti-angiogenic factors, anti-Alzheimer agents.

In some embodiments, the bioactive compound is a low to medium molecular weight compound (e.g. about 200 to 5000 Da, about 200 to about 1500 Da, preferably about 300 to about 1000 Da).

Exemplary cytotoxic drugs are particularly those which are used for cancer therapy. Such drugs include, in general, DNA damaging agents, anti-metabolites, natural products and their analogs, enzyme inhibitors such as dihydro folate reductase inhibitors and thymidylate synthase inhibitors, DNA binders, DNA alkylators, radiation sensitizers, DNA intercalators, DNA cleavers, microtubule stabilizing and destabilizing agents, topoisomerases inhibitors. Examples include but are not limited to platinum-based drugs, the anthracycline family of drugs, the vinca drugs, the mitomycins, the bleomycins, the cytotoxic nucleosides, taxanes, lexitropsins, the pteridine family of drugs, diynenes, the podophyllotoxins, dolastatins, maytansinoids, differentiation inducers, and taxols. Particularly useful members of those classes include, for example, auristatins, maytansines, maytansinoids, calicheamicins, dactinomycines, duocarmycins, CC1065 and its analogs, camptothecin and its analogs, SN-38 and its analogs; DXd, tubulysin M, cryptophycins, pyrrolobenzodiazepines and pyrrolobenzodiazepine dimers (PBDs), pyridinobenzodiazepines (PDDs) and indolinobenzodiazepines (IBDs) (cf. US20210206763A1), methotrexate, methopterin, dichloromethotrexate, 5-fluorouracil, DNA minor groove binders, 6-mercaptopurine, cytosine arabinoside, melphalan, leurosine, leurosideine, actinomycin, anthracyclines (doxorubicin, epirubicin, idarubicin, daunorubicin, PNU-159682 (cf. U.S. Pat. No. 10,288,745 B2.) and its analogs, mitomycin C, mitomycin A, caminomycin, aminopterin, tallysomycin, podophyllotoxin and; podophyllotoxin derivatives such as etoposide or etoposide phosphate, vinblastine, vincristine, vindesine, taxol, taxotere retinoic acid, butyric acid, N8-acetyl spermidine, staurosporin, colchicine, camptothecin, esperamicin, ene-diynes, and their analogues, hemiasterlin and its analogues.

Other exemplary drug classes are angiogenesis inhibitors, cell cycle progression inhibitors, P13K/m-TOR/AKT pathway inhibitors, MAPK signaling pathway inhibitors, kinase inhibitors, protein chaperones inhibitors, HDAC inhibitors, PARP inhibitors, Wnt/Hedgehog signaling pathway inhibitors, RNA polymerase inhibitors, and protein degraders (cf. https://pubs.acs.org/doi/10.1021/acschembio.0c00285).

Examples of auristatins include dolastatin 10, monomethyl auristatin E (MMAE), auristatin F, monomethyl auristatin F (MMAF), auristatin F hydroxypropylamide (AF HPA), auristatin F phenylene diamine (AFP), monomethyl auristatin D (MMAD), auristatin PE, auristatin EB, auristatin EFP, auristatin TP and auristatin AQ. Suitable auristatins are also described in U.S.; Publication Nos. 2003/0083263, 2011/0020343, and 2011/0070248; PCT Application; Publication Nos. WO09/117531, WO2005/081711, WO04/010957; WO02/088172 and WO01/24763, and U.S. Pat. Nos. 7,498,298; 6,884,869; 6,323,315; 6,239,104; 6,124,431; 6,034,065; 5,780,588; 5,767,237; 5,665,860; 5,663,149; 5,635,483; 5,599,902; 5,554,725; 5,530,097; 5,521,284; 5,504,191; 5,410,024; 5,138,036; 5,076,973; 4,986,988; 4,978,744; 4,879,278; 4,879,278; 4,816,444; and 4,486,414, the disclosures of which are incorporated herein by reference in their entirety.

Exemplary drugs include the dolastatins and analogues thereof including: dolastatin A (U.S. Pat. No. 4,486,414), dolastatin B (U.S. Pat. No. 4,486,414), dolastatin 10 (U.S. Pat. Nos. 4,486,444, 5,410,024, 5,504,191, 5,521,284, 5,530,097, 5,599,902, 5,635,483, 5,663,149, 5,665,860, 5,780,588, 6,034,065, 6,323,315), dolastatin 13 (U.S. Pat. No. 4,986,988), dolastatin 14 (U.S. Pat. No. 5,138,036), dolastatin 15 (U.S. Pat. No. 4,879,278), dolastatin 16 (U.S. Pat. No. 6,239,104), dolastatin 17 (U.S. Pat. No. 6,239,104), and dolastatin 18 (U.S. Pat. No. 6,239,104), each patent incorporated herein by reference in their entirety.

Exemplary maytansines, maytansinoids, such as DM-1 and DM-4, or maytansinoid analogs, including maytansinol and maytansinol analogs, are described in U.S. Pat. Nos. 4,424,219; 4,256,746; 4,294,757; 4,307,016; 4,313,946; 4,315,929; 4,331,598; 4,361,650; 4,362,663; 4,364,866; 4,450,254; 4,322,348; 4,371,533; 5,208,020; 5,416,064; 5,475,092; 5,585,499; 5,846,545; 6,333,410; 6,441,163; 6,716,821 and 7,276,497.

Other examples include mertansine and ansamitocin; Pyrrolobenzodiazepines (PBDs), which expressly include dimers and analogs, include but are not limited to those described in [Denny, Exp. Opin. Ther. Patents, 10 (4): 459-474 (2000)], [Hartley et al., Expert Opin Investig Drugs. 2011, 20 (6): 733-44], Antonow et al., Chem Rev. 2011, 111 (4), 2815-64].

Calicheamicins include, e.g. enediynes, esperamicin, and those described in U.S. Pat. Nos. 5,714,586 and 5,739,116.

Examples of duocarmycins and analogs include CC1065, duocarmycin SA, duocarmycin A, duocarmycin B I, duocarmycin B2, duocarmycin CI, duocarmycin C2, duocarmycin D, DU-86, KW-2189, adozelesin, bizelesin, carzelesin, seco-adozelesin. Other examples include those described in, for example, U.S. Pat. Nos. 5,070,092; 5,101,092; 5,187,186; 5,475,092; 5,595,499; 5,846,545; 6,534,660; 6,548,530; 6,586,618; 6,660,742; 6,756,397; 7,049,316; 7,553,816; 8,815,226; US20150104407; 61/988,011 filed May 2, 2014 and 62/010,972 filed Jun. 11, 2014; the disclosure of each of which is incorporated herein in its entirety.

Exemplary vinca alkaloids include vincristine, vinblastine, vindesine, and navelbine, and those disclosed in U.S. Publication Nos. 2002/0103136 and 2010/0305149, and in U.S. Pat. No. 7,303,749, the disclosures of which are incorporated herein by reference in their entirety.

Exemplary epothilone compounds include epothilone A, B, C, D, E, and F, and derivatives thereof. Suitable epothilone compounds and derivatives thereof are described, for example, in U.S. Pat. Nos. 6,956,036; 6,989,450; 6,121,029; 6,117,659; 6,096,757; 6,043,372; 5,969,145; and 5,886,026; and WO97/19086; WO98/08849; WO98/22461; WO98/25929; WO98/38192; WO99/01124; WO99/02514; WO99/03848; WO99/07692; WO99/27890; and WO99/28324; the disclosures of which are incorporated herein by reference in their entirety.

Exemplary cryptophycin compounds are described in U.S. Pat. Nos. 6,680,311; and 6,747,021; the disclosures of which are incorporated herein by reference in their entirety.

Exemplary platinum compounds include cisplatin, carboplatin, oxaliplatin, iproplatin, ormaplatin, tetraplatin.

Exemplary DNA binding or alkylating drugs include CC-1065 and its analogs, anthracyclines, calicheamicins, dactinomycines, mitomycines, pyrrolobenzodiazepines, and the like.

Exemplary microtubule stabilizing and destabilizing agents include taxane compounds, such as paclitaxel, docetaxel, tesetaxel, and carbazitaxel; maytansinoids, auristatins and analogs thereof, vinca alkaloid derivatives, epothilones and cryptophycins.

Exemplary topoisomerase inhibitors include camptothecin and camptothecin derivatives, camptothecin analogs and non-natural camptothecins, such as, for example, CPT-11, SN-38, topotecan, 9-aminocamptothecin, rubitecan, gimatecan, karenitecin, silatecan, lurtotecan, exatecan, DXd, diflometotecan, belotecan, lurtotecan and S39625. Other camptothecin compounds that can be used include those described in, for example, J. Med. Chem., 29:2358-2363 (1986); J. Med. Chem., 23:554 (1980); J. Med Chem., 30:1774 (1987).

Angiogenesis inhibitors include, but are not limited to, MetAP2 inhibitors, VEGF inhibitors, PIGF inhibitors, VGFR inhibitors, PDGFR inhibitors, MetAP2 inhibitors. Exemplary VGFR and PDGFR inhibitors include sorafenib, sunitinib and vatalanib. Exemplary MetAP2 inhibitors include fumagillol analogs, meaning compounds that include the fumagillin core structure.

Exemplary cell cycle progression inhibitors include CDK inhibitors such as, for example, BMS-387032 and PD0332991; Rho-kinase inhibitors such as, for example, AZD7762; aurora kinase inhibitors such as, for example, AZD1152, MLN8054 and MLN8237; PLK inhibitors such as, for example, BI 2536, BI6727, GSK461364, ON-01910; and KSP inhibitors such as, for example, SB 743921, SB 715992, MK-0731, AZD8477, AZ3146 and ARRY-520.

Exemplary P13K/m-TOR/AKT signalling pathway inhibitors include phosphoinositide 3-kinase (P13K) inhibitors, GSK-3 inhibitors, ATM inhibitors, DNA-PK inhibitors and PDK-1 inhibitors.

Exemplary P13 kinases are disclosed in U.S. Pat. No. 6,608,053, and include BEZ235, BGT226, BKM120, CAL263, demethoxyviridin, GDC-0941, GSK615, IC87114, LY294002, Palomid 529, perifosine, PF-04691502, PX-866, SAR245408, SAR245409, SF1126, Wortmannin, XL147 and XL765.

Exemplary AKT inhibitors include, but are not limited to AT7867.

Exemplary MAPK signaling pathway inhibitors include MEK, Ras, JNK, B-Raf and p38 MAPK inhibitors.

Exemplary MEK inhibitors are disclosed in U.S. Pat. No. 7,517,944 and include GDC-; 0973, GSKI 120212, MSC1936369B, AS703026, R05126766 and R04987655, PD0325901, AZD6244, AZD8330 and GDC-0973.

Exemplary B-raf inhibitors include CDC-0879, PLX-4032, and SB590885.

Exemplary B p38 MAPK inhibitors include BIRB 796, LY2228820 and SB 202190. Exemplary receptor tyrosine kinases inhibitors include but are not limited to AEE788 (NVP-AEE 788), BIBW2992 (Afatinib), Lapatinib, Erlotinib (Tarceva), Gefitinib (Iressa), AP24534 (Ponatinib), ABT-869 (linifanib), AZD2171, CHR-258 (Dovitinib), Sunitinib (Sutent), Sorafenib (Nexavar), and Vatalinib.

Exemplary protein chaperon inhibitors include HSP90 inhibitors. Exemplary inhibitors include 17AAG derivatives, BIIB021, BIIB028, SNX-5422, NVP-AUY-922 and KW-2478.

Exemplary HDAC inhibitors include Belinostat (PXD101), CUDC-101, Droxinostat, ITF2357 (Givinostat, Gavinostat), JNJ-26481585, LAQ824 (NVP-LAQ824, Dacinostat), LBH-589 (Panobinostat), MC1568, MGCD0103 (Mocetinostat), MS-275 (Entinostat), PCI-24781, Pyroxamide (NSC 696085), SB939, Trichostatin A and Vorinostat (SAHA). Exemplary PARP inhibitors include iniparib (BSI 201), olaparib (AZD-2281), ABT-888 (Veliparib), AG014699, CEP9722, MK 4827, KU-0059436 (AZD2281), LT-673, 3-aminobenzamide, A-966492, and AZD2461.

Exemplary Wnt/Hedgehog signalling pathway inhibitors include vismodegib, cyclopamine and XAV-939.

Exemplary RNA polymerase inhibitors include amatoxins. Exemplary amatoxins include alpha-amanitins, beta amanitins, gamma amanitins, eta amanitins, amanullin, amanullic acid, amanisamide, amanon, and proamanullin.

Exemplary cytokines include IL-2, IL-7, IL-10, IL-12, IL-15, IL-21, TNF. As non-limiting examples of particular drugs there may be mentioned Auristatins, Maytansinoids, PBDs, topoisomerase inhibitors, anthracyclines

In another embodiment, a combination of two or more different drugs as described above are used.

According to another embodiment, the bioactive compound may be selected from any synthetic or naturally occurring compounds comprising one or more natural and/or non-natural, proteinogenic and/or non-proteinogenic amino acid residues, such as in particular oligo- or polypeptides or proteins.

Other suitable examples of bioactive compounds are immunoglobulins such as antibodies, antibodies derivatives and active fragments thereof. Suitable examples in that regard are reported in the section D.3 above.

### D.4.2. Labelling Agents and Radionuclides

Labeling agents which may be used as biopolymer within the meanings of the present invention can comprise any type of label known in the art.

Examples are dyes (e.g. fluorescent, luminescent, or phosphorescent dyes (e.g. fluorescent, luminescent, or phosphorescent dyes), such as dansyl, coumarin, fluorescein, acridine, rhodamine, silicon-rhodamine, BODIPY, or cyanine dyes), molecules able to emit fluorescence upon contact with a reagent, chromophores (e.g., phytochrome, phycobilin, bilirubin, etc.), radiolabels (e.g. radioactive forms of hydrogen, fluorine, carbon, phosphorous, sulphur, or iodine, such as tritium, fluorine-18, carbon-11, carbon-14, phosphorous-32, phosphorous-33, sulphur-33, sulphur-35, indium-111, iodine-123, or iodine-125), MRI-sensitive spin labels, affinity tags (e.g. biotin, His-tag, Flag-tag, strep-tag, sugars, lipids, sterols, PEG-linkers, benzylguanines, benzylcytosines, or co-factors), polyethylene glycol groups (e.g., a branched PEG, a linear PEG, PEGs of different molecular weights, etc.), photocrosslinkers (such as p-azidoiodoacetanilide), NMR probes, X-ray probes, pH probes, IR probes, resins, solid supports and bioactive compounds as defied above.

In some embodiments, exemplary dyes can include an NIR contrast agent that fluoresces in the near infrared region of the spectrum. Exemplary near-infrared fluorophores can include dyes and other fluorophores with emission wavelengths (e.g., peak emission wavelengths) between about 630 and 1000 nm, e.g., between about 630 and 800 nm, between about 800 and 900 nm, between about 900 and 1000 nm, between about 680 and 750 nm, between about 750 and 800 nm, between about 800 and 850 nm, between about 850 and 900 nm, between about 900 and 950 nm, or between about 950 and 1000 nm. Fluorophores with emission wavelengths (e.g., peak emission wavelengths) greater than 1000 nm can also be used in the methods described herein. In some embodiments, exemplary fluorophores include 7-amino-4-methylcoumarin-3-acetic acid (AMCA), TEXAS RED™ (Molecular Probes, Inc., Eugene, Oreg.), 5-(and -6)-carboxy-X-rhodamine, lissamine rhodamine B, 5-(and -6)-carboxyfluorescein, fluorescein-5-isothiocyanate (FITC), 7-diethylaminocoumarin-3-carboxylic acid, tetramethylrhodamine-5-(and -6)-isothiocyanate, 5-(and -6)-carboxytetramethylrhodamine, 7-hydroxycoumarin-3-carboxylic acid, 6-[fluorescein 5-(and -6)-carboxamido]hexanoic acid, N-(4,4-difluoro-5,7-dimethyl-4-bora-3a,4a diaza-3-indacenepropionic acid, eosin-5-isothiocyanate, erythrosin-5-isothiocyanate, and CASCADE™ blue acetylazide (Molecular Probes, Inc., Eugene, Oreg.) and ATTO dyes.

Further labelling agents are 111-Indium, 64-Copper, 67-Copper, 124-lodine, 227-Thorium, 188-Rhenium, 177-Lutetium, 89-Zirkonium, 131-Iod, 68-Gallium, 99m-Technecium, 225-Actinium, 213-Bismut, 90-Ytrium and 212-Plumbum.

### D.4.3. Chelators

Lists of typically applicable chelators and their short names are given below. Corresponding salts thereof are also applicable.

Acetyl acetone (ACAC), ethylene diamine (EN), 2-(2-aminoethylamino) ethanol (AEEA), diethylene triamine (DIEN), iminodiacetate (IDA), triethylene tetramine (TRIEN), triaminotriethylamine, nitrilotriacetate (NTA) and its saltslike Na3NTA or FeNTA, ethylenediaminotriacetate (TED), ethylenediamine tetraacetate (EDTA) and its salts like Na2EDTA and CaNa2EDTA, diethylene triaminpentaacetate (DTPA), 1,4,7,10-ztetraazacyclododecane-1,4,7,10-tetraacetate (DOTA), 1,4,7-triazacyclononane-1,4,7-triacetic acid (NOTA), Oxalate (OX), tartrate (TART), citrate (CIT), dimethylglyoxime (DMG), 8-hydroxyquinoline, 2,2′-bipyridine (BPY), 1,10-phenanthroline (PHEN), dimercapto succinic acid (DMSA), 1,2-bis(diphenylphosphino) ethane (DPPE), sodium salicylate, methoxy salicylates, British anti-Lewisite or 2,3-dimercaprol (BAL), meso-2,3-dimercaptosuccinic acid (DMSA); Siderophores secreted by microorganisms, as for example desferrioxamine or deferoxamine B, also known as Deferral (Novartis), produced by Streptomyces spp.; deferoxamine (DFO), a trihydroxamic acid secreted by Streptomyces pilosus; phytochemicals like curcuminoids and derivatives of mugineic acid, like 3-hydroxy-mugineic acid and 2′-deoxy-mugineic acid; synthetically produced chelators, like Ibuprofen; derivatives of catechol, hydroxamate and hydroxypyridinone, like hydroxamate desferal and hydroxypyridinone deferiprone; deferiprone (L1 or 1,2-dimethyl-3-hydroxypyrid-4-one); D-penicillamine (DPA or D-PEN) which is β-β-dimethylcysteine or 3-mercapto-D-valine; tetraethylenetetraamine (TETA) or trientine and its two major metabolites N1-acetyltriethylenetetramine (MAT) and N1,N10-diacetyltriethylenetetramine (DAT); hydroxyquinolines; clioquinol, which is a halogenated derivative of 8-hydroxyquinoline; and 5,7-dichloro-2-[(dimethylamino)methyl]quinolin-8-ol (PBT2).

### D.5. Pharmaceutical Composition

The stabilized composition (i.e. the stabilized active ingredients, in particular biopolymers) of this invention are generally given as “pharmaceutical compositions” comprised of a “therapeutically” and/or “prophylactically effective amount” or a “diagnostically” effective amount of at least one such active ingredient or its pharmaceutically acceptable salt and optionally at least one pharmaceutically acceptable excipient.

Thus the term “pharmaceutical composition” according to a particular embodiment of the present invention designates a stabilized liquid composition comprising or essentially consisting of at least one pharmaceutically active biopolymer compound (i.e. the active ingredient) and at least one stabilizing EO/BuO block copolymer as described herein in a liquid, pharmaceutically acceptable medium. A dried powder of such liquid preparation can be obtained by lyophilization or any other suitable drying method that is typically applied.

Said pharmaceutical compositions may be delivered via suitable routes of administration such as via oral, rectal, transmucosal, topical, ophthalmic, otologic, or intestinal administration; parenteral delivery, including intramuscular, subcutaneous, intramedullary injections, as well as intrathecal, direct intraventricular, intravenous, intraperitoneal, intranasal, or intraocular injections, as the case may be.

Depending on the nature or the mode of administration and dosage form said composition said at least one additional pharmaceutical excipient may be different

An “excipient” is a substance formulated alongside the active ingredient and is included for different purpose, as for example for long-term stabilization, bulking up solid formulations that contain potent active ingredients in small amounts (thus often referred to as “bulking agents”, “fillers”, or “diluents”), or to confer a therapeutic enhancement on the active ingredient in the final dosage form, such as for example facilitating drug absorption, reducing viscosity, or enhancing solubility. Excipients can also be useful in the manufacturing process of the pharmaceutical composition, to aid in the handling of the active substance concerns such as by facilitating powder flowability or non-stick properties, in addition to aiding in vitro stability such as prevention of denaturation or aggregation over the expected shelf life. The selection of appropriate excipients not only depends upon the route of administration and the dosage form, but also on the particular active ingredient and other factors.

Excipients may be selected from the following classes: immunological adjuvants, antiadherents, binders, coatings, colours, disintegrant, flavours, glidants, lubricants, preservatives, sorbents, sweeteners, and vehicles

Non limiting examples of excipients comprise diluents, preserving agents, stabilizers, emulsifying agents, like emulsifying polymers, such as polysorbates or poloxamers, antioxidants, as for example chemical compounds, like epigallocatechin-3-O-gallate, lycopene, ellagic acid, coenzyme Q₁₀, indole-3-carbinol, genistein, quercetin, ascorbic acid, glutathione, melatonin, catechin, taurine, captopril, gallic acid, N-acetyl cysteine, α-lipoic acid, BHT, tocopherols and tocotrienols, or enzymes like superoxide dismutase and catalase; anti-irritants, chelating agents and stabilizing salts, such as chlorides, sulfates, phosphates, diphosphates, hydrobromides and nitrates, suspending agents, antibacterial agents or antifungal agents. Further, buffering agents such as buffering systems of low molecular weight organic acids together with the respective salts, or inorganic buffering substances, such as phosphate buffers, can be used, Further suitable ingredients are also known from relevant pharmacological standard literature. Also the proportion of the various components will vary depending on the nature of the specific component used and is generally known to the person skilled in the art (Remington's Pharmaceutical science (“Handbook of Pharmaceutical Excipients”, 2nd Edition, (1994), Edited by A Wade and PJ Weller or in Remington's Pharmaceutical Sciences, Mack Publishing Co. (A. R Gennaro edit. 1985).

A pharmaceutical composition as used herein may be presented in the form of a “dosage form” or “unit dose” and may comprise one or more stabilized liquid composition, or essentially dry biopolymer composition comprising at least one pharmaceutically active biopolymer compound and at least one stabilizing EO/BuO block copolymer as described herein. Thus, a pharmaceutical composition as used herein could, for example, provide two active agents admixed together in a unit dose or provide two active agents combined in a dosage form wherein the active agents are physically separated.

Furthermore, one may administer said pharmaceutical composition in a targeted drug delivery system, for example, in a liposome coated with endothelial cell-specific antibody.

The pharmaceutical compositions of the present invention may be manufactured in a manner that is itself known, e.g., by means of conventional mixing, dissolving, emulsifying, encapsulating, entrapping or combinations thereof. Proper formulation is dependent upon the route of administration chosen.

The phrase “pharmaceutically acceptable” is employed herein to refer to those compounds, materials, compositions, and/or dosage forms which are, within the scope of sound medical judgment, suitable for use in contact with the tissues of patients without excessive toxicity, irritation, allergic response, or other problem or complication commensurate with a reasonable risk/benefit ratio.

The invention includes all “pharmaceutically acceptable salt forms” of the active ingredient. Pharmaceutically acceptable salts are those in which the counter ions do not contribute significantly to the physiological activity or toxicity of the compounds and as such function as pharmacological equivalents. These salts can be made according to common organic techniques employing commercially available reagents. Some anionic salt forms include acetate, acistrate, besylate, bromide, chloride, citrate, fumarate, glucouronate, hydrobromide, hydrochloride, hydroiodide, iodide, lactate, maleate, mesylate, nitrate, pamoate, phosphate, succinate, sulfate, tartrate, tosylate, and xinofoate. Some cationic salt forms include ammonium, aluminum, benzathine, bismuth, calcium, choline, diethylamine, diethanolamine, lithium, magnesium, meglumine, 4-phenylcyclohexylamine, piperazine, potassium, sodium, tromethamine, and zinc.

A “therapeutically effective amount” and/or “prophylactically effective amount” means an amount effective, when administered to a human or non-human patient, to provide any therapeutic and/or prophylactic benefit. More particularly, a “therapeutically effective amount” is an amount of an active ingredient disclosed herein or a combination of two or more such active ingredients, which inhibits, totally or partially, the progression of the condition or alleviates, at least partially, one or more symptoms of the condition.

A “diagnostically effective amount” means an amount effective to allow obtaining from the patient a diagnostically valuable information on status or progression of a disease state.

A therapeutic benefit may be an amelioration of symptoms of a diseased patient, e.g., an amount effective to decrease the symptoms of a diseased patient. In certain circumstances a patient may not present symptoms of a condition for which the patient is being treated. Thus, a prophylactically effective amount of a compound is also an amount sufficient to provide a significant positive effect on any indicia of a disease, disorder or condition e.g. an amount sufficient to significantly reduce the frequency and severity of disease symptoms to occur.

A therapeutically effective amount can also be an amount, which is prophylactically effective.

A “patient” as used herein means human or non-human, in particular human, animals.

A “dosage form” is any unit of administration (“unit dose”) of one or more active agents as described herein.

The term “treating” or “treatment” refers to: (i) preventing a disease, disorder or condition from occurring in a patient which may be predisposed to the disease, disorder and/or condition but has not yet been diagnosed as having it; (ii) inhibiting the disease, disorder or condition, i.e., arresting its development; and (iii) relieving the disease, disorder or condition, i.e., causing regression of the disease, disorder and/or condition. In particular it encompasses a prophylactic or therapeutic treatment or combinations thereof.

“Frequency” of dosage may vary depending on the compound used and the particular type of infection treated. A dosage regimen of once per day is possible. Dosage regimens in which the active agent is administered for several times daily, as for example 2 to 10 times, like 2, 3, 4, 5, 6, 7, 8, 9 or 10 times may occasionally be more helpful.

It will be understood, however, that the specific dose level and frequency for any particular patient will depend upon a variety of factors including the activity of the specific compound employed, the age, body weight, general health, sex, diet, time of administration, route of administration, rate of excretion, drug combination and the severity of the particular disease in the patient undergoing therapy. Patients may generally be monitored for therapeutic or prophylactic effectiveness using assays suitable for the condition being treated or prevented, which will be familiar to those of ordinary skill in the art.

Particular examples of pharmaceutical composition according to the present invention are liquid form preparations such as solutions, suspensions, and emulsions and comprise, beside the block copolymer according to the present invention, a therapeutically effective amount of biopolymer component as defined above, optionally together with at least one further pharmaceutically acceptable excipient as defined above and may be administered through any suitable route.

Further examples of pharmaceutical composition according to the present invention are solid form preparations such as powders, tablets, pills, capsules, cachets, suppositories, and dispersible granules.

The following example serve for a better understanding of the present invention without limiting its scope.

### Experimental Part

### A. MATERIAL AND METHODS

### A.1 Chemicals and Buffers

Unless stated otherwise all chemicals as applied were of analytical grade and obtained from commercial sources.

APIs were obtained from commercial sources.

Bovine immunoglobulin (IgG) captured from pooled bovine plasma using a chromatographic method (MPBio, Cat-No: 08641402, Lot 21040)

### A.2 Analytical Methods

The HPLC measurement was done by gradient elusion using a RP-C18 column (Chromolith®, HighResolution, RP-18 endcapped, 100-4.6 mm, Merck KGaA) which was kept at 25° C. The mobile phase composed of solvent A (water, 0.1% phosphoric acid) and solvent B (acetonitrile, 0.1% phosphoric acid) was at a flow rate of 1.5 ml/min. The samples were detected and quantified using a DAD detector. The herein used HPLC device was the 1260 Infinity II from Agilent Technologies.

¹H-NMR spectra were measured in CDCl₃ with a Bruker AVANCE III 500 MHz spectrometer.

### A.3 General Protocol for Determining of Ethylene Oxide Content by 1H-NMR

Weight percent of ethylene oxide (wt % EO) in the claimed polymers was determined by ¹H-NMR spectroscopy. In 1H-NMR spectroscopy, the integral of each peak is proportional to the molar concentration of the protons being analysed.

For butylene oxide and ethylene oxide block copolymers (Butronics), the CH₃ groups from polymerized butylene oxide give a triplett signal at 8=0.95 ppm.

The ethylene oxide repeating group-O—CH₂—CH₂—O— and the butylene oxide repeating group-O—CH₂—CH (Et)-O— give a broad multiplett from 8=3.0 to 4.2 ppm, whereas the contribution of each ethylene oxide repeating group is 4 protons (two CH₂ groups), and each butylene oxide group contributes with 3 protons (one CH and one CH₂ group) to the signal.

To determine the mol % of ethylene oxide and the wt % of ethylene oxide following calculations are done:

Integral at 8=0.95 ppm is set as 3 protons is [Area BuO] Integral at 8=3.0 to 4.2 ppm is [Area EO+BuO]

$$[ Area EO ] = [ Area EO + BuO ] - [ Area BuO ]$$

$$mol \% EO = ( [ Area EO ] / 4 ) / ( ( [ area EO ] / 4 ) + ( [ Area BuO ] / 3 ) ) * 100$$

$$mol \% BuO = ( [ Area BuO ] / 3 ) / ( ( [ area EO ] / 4 ) + ( [ Area BuO ] / 3 ) ) * 100$$

From mol % EO and mol % BuO the weight percent of ethylene oxide wt % EO is calculated with molecular mass of ethylene oxide (44.05 g/mol) and molecular mass of butylene oxide (72.11 g/mol):

$$wt \% EO = mol \% EO * 44.05 / ( ( mol \% EO * 44.05 ) + ( mol \% BuO * 72.11 ) * 100$$

### A.4 General Protocol for IgG Aggregation Assay

The test is performed with commercial bovine immunoglobulin (IgG) captured from pooled bovine plasma using a chromatographic method. For every test three references are tested in addition to the samples: IgG without surfactant (blank), IgG with PS20 (positive control), and IgG with P188 (negative control).

1 g IgG is dissolved in 50 mL 20 mM histidine buffer pH 6 by careful manual shaking. The IgG solution is stored overnight in the refrigerator at 4° C. 40 mg of each surfactant is dissolved in 10 mL 20 mM histidine buffer pH 6 and stored overnight in the refrigerator at 4° C.

To avoid particle contamination, all following steps are carried out under the clean bench. The surfactant solutions are diluted 1:100 with 20 mM histidine buffer pH 6 to gain a concentration of 0.004%.

The IgG solution and the surfactant solutions are filtered through a PVDF syringe filter with 0.22 μm pore size. For each surfactant and for the blank 0.75 mL of the surfactant (histidine buffer for blank) and 0.75 mL of the IgG solution are added to five glass vials per sample. This leads to an IgG concentration of 10 mg/ml and a surfactant concentration of 0.002%. Four glass vials per sample are shaken at 200 rpm for 15 hours (T15) on an IKA HS 501 horizontal shaker.

The remaining glass vial (TO) is inverted 3 times, diluted 1:10 in a deep-well plate with histidine buffer pH 6 and the particle count is quantified by micro-flow imaging (MFI) in the size range from 1 μm to 300 μm.

For all samples (TO and T15) the particle count is quantified by micro-flow imaging (MF15200 by ProteinSimple). The samples are prepared for the measurement by pipetting 0.1 mL of each sample into a 96 deep-well plate, followed by 0.1 mL 200 mM histidine buffer and 0.8 mL HPLC grade water. All samples are measured in duplicates.

For the analysis 0.6 mL of the sample are used and the equivalent circular diameter (ECD) of the particles is determined in a range from 1 μm to 300 μm after removal of edge, stuck and slow particles.

The particle count for PS20 ranges from ca. 150 to 7,800 particles and for P188 from ca. 10,200 to 92,000 particles in 0.6 mL diluted sample, depending on the IgG batch that is used. For a good differentiation between different surfactants, an IgG batch should be used that, with the described protocol and in absence of surfactant (blank), results in at least 20,000 particles in 0.6 mL diluted sample. While the particle count changes for different IgG batches, the trend between the different surfactants is consistent.

For analysis, the particle count of the blank is set in relation to the aggregation of the samples with surfactant (the particle count of the blank is set as 100% aggregation).

### A.5 General Protocol for Water Solubility Assay

To determine a polymer solubility of a 10 wt % solution in a 100 ml glass flask 7 g polymer (100%) and 63 g distilled water are placed at room temperature. The mixture is stirred with a magnetic stirrer until polymer is completely dissolved. To determine solubility at other concentrations, polymer solutions with various polymer content are prepared in a similar way.

### A.6 General Protocol for Surface Tension Assay

For the characterization of the surface activity of synthesized polymers, samples were dissolved in deionized water at a concentration of 1 g/L and subsequently diluted to 0.1 g/L.

After stirring for 2 h and complete dissolution, the surfactant solutions were filled into a disposable syringe, which was then mounted on a Drop Shape Analyser (DSA) 100 drop shape tensiometer from Krüss (Hamburg, Germany).

Static surface tension was measured at 0.1 g/L by the pendant drop technique, where a free-hanging droplet of surfactant solution (typical volumes: 1-10 μL depending on surface tension) is generated at the outlet of the syringe. Then, a two-dimensional projection of the hanging droplet is acquired by an integrated camera system, from which the drop contour is determined via image analysis utilizing the instrument software Advance 1.9.2.

Fitting of the drop contour based on the Young-Laplace equation (C. Samuel et al., Polymer Testing, 2019, 78, 105995) yields the desired values for the surface tension.

The density of the solutions required for evaluation was assumed to be that of pure water.

Surface tension was monitored over a period of 5 minutes, and the measured values were averaged. All measurements were performed at 23° C.

### A.7 General Protocol for Haemolysis Assay

The principle of an RBC-test is described by Hoover (D. M: Hoover et al., Fundamental and Applied Toxicology 1990, 14, 589-597.) and Pape (W. J. W. Pape et al., Molecular Toxicology 1987, 1:525-536.). The test is based on the integrity of the red blood cell (RBC) membrane and determines the degree of hemolysis after agitation of a cell suspension at different test compound concentrations. In case of RBC membrane damage due to the test substance, hemoglobin is released via the disrupted cell membrane into the test solution. The free hemoglobin concentration in the test solution is measured as correlate for the RBC membrane damage caused by the test substance.

Preparation of red blood cell (RBC) suspension: RBCs from EDTA blood of human blood donors were isolated by centrifugation and were washed three times with phosphate buffered saline plus glucose (PBS/glucose) to remove traces of plasma and the bulk of white blood cells. The washed RBCs were diluted with (PBS/glucose) and were adjusted to an approximate 2% RBC suspension.

Test procedure: A test solution of the polymer of 1.33% (=1.33 g/10 ml, i.e. final concentration in test 100 mg/ml) was prepared in PBS/glucose and adjusted to pH 7.4. Further test solutions (final concentration of polymer of 10 and 1 mg/ml) were made by dilution with PBS/glucose. One volume of the RBC suspension was added to three volumes of test solutions, resulting in final test compound concentrations of 100, 10, and 1 mg/ml. The assay mixtures were incubated at room temperature with shaking by an Eppendorf mixer (Model 5432; at 1000 rpm) for 60 minutes.

After incubation the samples were centrifuged to sediment remaining intact RBCs and membrane fragments.

Released free haemoglobin in the supernatant as a degree of hemolysis was determined spectrophotometrically at 540 nm.

Results were compared to totally lysed RBCs in distilled water (100% hemolysis) and to a fragility control with PBS/glucose (spontaneous, no substance related hemolysis). All samples were evaluated in triplicates.

Tested polymers were classified according to following scheme:
low: <10% hemolysis at 100 mg/ml
medium: <10% hemolysis at 10 mg/ml
high: ≥hemolysis at 10 mg/ml

### A.8 General Protocol for the Determination of Small Molecule Solubilisation Ability by Butronics

A 10% (m/m) aqueous solution of the test substances (butronics as well as reference materials Kolliphor EL and HS15) is prepared in phosphate buffer pH 7.4. For each test substance and reference three 5 ml brown glass ampoules with screw caps are filled with 5 g of the prepared aqueous solution.

The respective API (as for example Fenofibrate/Itraconazole/Nilotinib) to be analyzed is then added (about 30 mg-500 mg per 5 g of 10% test substance solution, enough that the added powder doesn't dissolve anymore) in order to obtain a supersaturated solution.

After stirring for 72 h at room temperature, each sample is filtered through a 0.22 μm PVDF filter and the concentration of the respective API in the filtrate is quantified by HPLC.

The average of the 3 replicates per API is calculated and the obtained concentrations are put in relation to Kolliphor EL, which is set to 100%.

For Table 7 in item C.6, the average of the results of Fenofibrate, Itraconazole and Nilotinib is shown.

### A.9 General Protocol for the Determination of Cloud Point

Cloud points are determined according to DIN EN 1890, method A

In a 100 ml glass flask 0.4 g polymer (100%) and 39.6 g dist. water are placed at room temperature. The mixture is stirred with a magnetic stirrer until polymer is completely dissolved. Ca. 30 ml of this solution are filled in a test glass. A thermometer is placed in the test glass to monitor the temperature. The test glass is heated slowly with a hot air dryer until a persistent turbidity is observed.

Cloud point is determined as the temperature, which induces a change from a clear to a turbid solution. Values of 20° C. indicates the solution is turbid at room temperature. Cloud point of >95° C. means no turbidity is observed up to 95° C.

### A.10 General Protocol for the Preparation of PLGA Nanoparticles

PLGA nanoparticles are prepared via nanoprecipitation. A solution of 50 mg/mL PLGA (Resomer® RG502H) in acetone is prepared. 0.5 mL of this solution is slowly injected into 9.5 mL of HPLC grade water while strongly stirring the water. For the injection the tip of an Eppendorf tip is dipped into the strongly stirring water and the PLGA solution slowly ejected from the tip. The final acetone concentration in the resulting slightly turbid suspension is 5% and the PLGA concentration is 2.5 mg/mL. The particle size of the resulting PLGA particles (measured by DLS with a Malvern Zetasizer Nano S) is around 230 nm with a PDI of circa 0.1.

For each surfactant (Poloxamer P188 and the butronics) a 5% (w/w) solution in water is prepared by adding 9.5 mL of HPLC grade water to 0.5 g of surfactant and shaking at 1000 rpm for at least 20 min on a Thermoshaker until all surfactant is fully dissolved. Then the solution is filtered with a 0.2 μm PVDF syringe filter.

For each surfactant 0.2 mL of the 5% surfactant solution is added to 1.8 mL of the PLGA suspension. The resulting suspensions are briefly mixed and then incubated for ca. 5 min. Particle size and dispersity of the undiluted suspensions are measured by DLS (Malvern Zetasizer Nano S) following manufacturer's instructions. Then the solutions are transferred into 2 mL Eppendorf tubes and centrifuged at 14800 rpm for 20 min. At the bottom of the tube white pellets form. The supernatants are removed, and the pellets resuspended in 2 mL HPLC grade water each. All samples are briefly (a few seconds) vortexed and then particle size and dispersity of the undiluted suspensions are measured again (Malvern Zetasizer Nano S, following manufacturer's instructions).

### A.11 General Protocols for Further Biopolymer Aggregation Assays

### A.11.1 Protocol for β-Casein

The test is performed with commercial β-Casein extracted from bovine milk and executed according to the protocol depicted in item A.4. The final concentration of the protein is 3.125 mg/mL.

### A.11.2 Protocol for Lysozyme

The test is performed with commercial Lysozyme and executed based on the protocol depicted in item A.4.

0.1 g Lysozyme is dissolved in 50 mL phosphate buffered saline (PBS) pH 7.4 by careful manual shaking. The Lysozyme solution is stored overnight in the refrigerator at 4° C. The surfactant solutions are prepared as described in item A.4, whereby PBS is used instead of histidine buffer.

For each surfactant and for the blank, 0.4 mL of the surfactant (PBS for blank) and 0.4 mL Lysozyme solution are added to four glass vials per sample. This leads to a Lysozyme concentration of 1 mg/mL and a surfactant concentration of 0.002%. The glass vials are shaken at 500 rpm for 24 hours at room temperature in a MaxQ™ 6000 incubation shaker from Thermo Fisher Scientific.

For all samples, the particle count is quantified by micro-flow imaging (MFI5200 from Protein Simple). The sample preparation and measurement are executed as depicted in item A.4.

For analysis, the particle count of the blank is set in relation to the aggregation of the samples with the surfactant (the particle count of the blank is set as 100% aggregation).

### A.11.3 Protocol for Thrombin

The test is performed with commercial Thrombin and is executed according to the protocol depicted in item A.4. The final concentration of the protein is 1 mg/ml and the final volume in the vials is 0.8 mL.

### A.11.4 Protocol for Carbonic Anhydrase

The test is performed with commercial carbonic anhydrase and is executed according to the protocol depicted in item A.4. The final concentration of the protein is 1 mg/mL and the final volume in the vials is 0.8 mL. The vials are shaken at 300 rpm for 4 hours on an IKA HS 501 horizontal shaker.

A.11.5 Protocol for IgG Extracted from Human Serum

The test is performed with commercial IgG extracted from human serum and is executed according to the protocol depicted in item A.4. The final concentration of the protein is 1 mg/mL and the final volume in the vials is 0.8 mL. The vials are shaken at 300 rpm for 4.5 hours on an IKA HS 501 shaking board.

A.11.6 Protocol for Monoclonal Antibody 4 (mAb4)

The test is performed with a recombinant humanized IgG1 monoclonal antibody (mAb4). For every test, four references are tested in addition to the samples: mAb4 without surfactant and stored at room temperature without shaking (unstressed mAb4), mAb4 without surfactant and stressed by shaking (blank), mAb4 with PS20 (positive control), and mAb4 with P188 (negative control).

The antibody is already dissolved in 20 mM histidine buffer pH 6 and stored in the freezer at −80° C. After thawing in the refrigerator at 4° C., the antibody solution is further diluted to a concentration of 2 mg/mL. The surfactant solutions are prepared as described in item A.4.

For each surfactant, the blank and the unstressed sample, 0.4 mL of the surfactant (histidine for blank and unstressed sample) and 0.4 mL diluted mAb4 solution are added to four glass vials per sample. This leads to a mAb4 concentration of 1 mg/ml and a surfactant concentration of 0.002%. The glass vials are shaken at 300 rpm for 15 hours at room temperature on an IKA HS 501 shaking board, whereas the vials with unstressed mAb4 are stored at room temperature without shaking.

As the blank samples are very turbid, those samples are not further analyzed by micro-flow imaging, whereas the remaining samples are analyzed as described in item A.4.

For analysis, the particle count of the samples is set in relation to the aggregation of the unstressed mAb4 (the particle count of the unstressed mAb4 is set as 100% aggregation).

A.11.7 Protocol for Monoclonal Antibody 3 (mAb3)

The test is performed with a recombinant humanized IgG2 monoclonal antibody (mAb3). For every test, four references are tested in addition to the samples: mAb3 without surfactant and stored at room temperature without shaking (unstressed mAb3), mAb3 without surfactant and stressed by shaking (blank), mAb3 with PS20 (positive control), and mAb4 with P188 (negative control).

The antibody is already dissolved in 20 mM histidine buffer pH 6 and stored in the freezer at −80° C. After thawing in the refrigerator at 4° C., the antibody solution is further diluted to a concentration of 2 mg/mL. 40 mg of each surfactant is dissolved in 10 mL 20 mM histidine buffer pH 6 with 600 mM NaCl and stored overnight in the refrigerator at 4° C.

To avoid particle contamination, all following steps are carried out under the clean bench. The surfactant solutions are diluted 1:100 with 20 mM histidine buffer pH 6 with 600 mM NaCl to gain a concentration of 0.004%.

For each surfactant, the blank and the unstressed sample, 0.4 mL of the surfactant (histidine with 600 mM NaCl for blank and unstressed sample) and 0.4 mL diluted mAb3 solution are added to four glass vials per sample. This leads to a mAb3 concentration of 1 mg/mL, a surfactant concentration of 0.002% and a NaCl concentration of 300 mM. The glass vials are shaken at 400 rpm for 4.5 to 5 hours at room temperature in a MaxQ™ 6000 incubation shaker from Thermo Fisher Scientific, whereas the vials with unstressed mAb3 were stored at room temperature.

All further analyzation is carried out as described in item A.4.

### B. SYNTHESIS EXAMPLES

Exemplary butronics according to the invention (Test samples No. 1 to 10) as well as comparative Butronics (CE1 to CE5) are characterized by the following general formula 1a

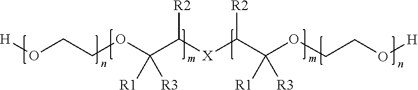

(1a)

All polymers were synthetized by applying synthetic protocols similar to those described in synthesis example 1 (X derived from 1,4-butane diol) below.

Molecular weights given are calculated from the used molar ratio of starting materials. The ethylene oxide content (wt % EO) was determined following the protocol described above (see item A.3).

### Synthesis Example 1:1,4-Butane Diol, Butoxylated with 20 Mole 1,2-Butylene Oxide and Ethoxylated with 34 Mole Ethylene Oxide (Synthesis Method A)

Step a: Synthesis of 1,4-Butane Diol, Butoxylated with 20 Mole 1,2-Butylene Oxide

In a 21 autoclave 90.12 g 1,4-butane diol and 3.1 g potassium tert. butoxide were placed and the reactor was purged three times with nitrogen. The mixture was heated to 140° C. 1440.0 g 1,2-butylene oxide was added within 20 hours. To complete the reaction, the mixture was allowed to post-react for additional 10 hours at 140° C. The reaction mixture was stripped with nitrogen and volatile compounds were removed in vacuo at 90° C. for 2 hours. 1528.0 g of a light orange oil was obtained.

¹H-NMR in CDCl₃ confirmed the complete conversion to the expected polymer.

Step b: Synthesis of 1,4-Butane Diol, Butoxylated with 20 Mole 1,2-Butylene Oxide and Ethoxylated with 34 Mole Ethylene Oxide

In a 2 l autoclave 404.5 g 1,4-butane diol, butoxylated with 20 mole 1,2-butylene oxide (from step a) and 1.6 g potassium tert. butoxide were placed and the reactor was purged three times with nitrogen. The mixture was heated to 110° C. 395.5 g ethylene oxide was added within 20 hours. To complete the reaction, the mixture was allowed to post-react for additional 10 hours at 140° C. The reaction mixture was stripped with nitrogen, and 1.3 g acetic acid was added for neutralization. Volatile compounds were removed in vacuo at 90° C. for 2 hours. 800.0 g of a beige solid was obtained.

¹H-NMR in CDCl₃ confirmed the complete conversion to the expected polymer. Hydroxy value was measured to be 37.2 mg KOH/g, water was content 0.1%.

### Synthesis Example 2:1,4-Butane Diol, Butoxylated with 24 Mole 1,2-Butylene Oxide and Ethoxylated with 96 Mole Ethylene Oxide-Synthesis Step 1 in Presence of Water (Synthesis Method B)

Step a: Synthesis of 1,4-Butane Diol, Butoxylated with 10 Mole 1,2-Butylene Oxide—In Presence of Water—

In a 2 l autoclave 85.6 g 1,4-butane diol and 3.4 g potassium hydroxide (50% in water) were placed and the reactor heated to 100° C. The reactor was purged three times with nitrogen. The mixture was heated to 140° C. 771.0 g 1,2-butylene oxide was added within 6 hours. To complete the reaction, the mixture was allowed to post-react for additional 3 hours at 140° C. The reaction mixture was stripped with nitrogen and volatile compounds were removed in vacuo at 90° C. for 2 hours. 852.0 g of a light orange oil was obtained.

¹H-NMR in CDCl₃ confirmed the complete conversion to the expected polymer. Hydroxy value was measured to be 130.0 mg KOH/g, water content was 0.13 wt %.

Step b: Synthesis of 1,4-Butane Diol, Butoxylated with 24 Mole 1,2-Butylene Oxide and Ethoxylated with 96 Mole Ethylene Oxide

In a 2 l autoclave 145.8 g 1,4-butane diol, butoxylated with 10 mole 1,2-butylene oxide (from step a) and 1.6 g potassium hydroxide (50% in water) were placed. Vacuum was applied (<25 mbar) and the mixture was heated to 125° C. The mixture was stirred for 2.5 hours at 125° C. and <25 mbar vacuum. Vacuum was broken with nitrogen. The mixture was heated to 140° C. 181.4 g butylene oxide was added within 2 hours. To complete the reaction, the mixture was allowed to post-react for 6 hours. 761.0 g ethylene oxide was added within 6 hours. To complete the reaction, the mixture was allowed to post-react for additional 2 hours at 140° C. The reaction mixture was stripped with nitrogen, and 2.5 g phosphoric acid (75% in water) was added for neutralization. Volatile compounds were removed in vacuo at 90° C. for 2 hours. 0.10 g alpha-tocopherol was added and the mixture was stirred for 0.25 hours. After cooling, 1081.0 g of a beige solid was obtained.

¹H-NMR in CDCl₃ confirmed the complete conversion to the expected polymer. Hydroxy value was measured to be 24.8 mg KOH/g, water was content 0.1%.

### Synthesis Example 3: Isosorbid, Butoxylated with 24 Mole 1,2-Butylene Oxide and Ethoxylated with 96 Mole Ethylene Oxide (Synthesis Method C)

Step a: Synthesis of Isosorbid, Butoxylated with 10 Mole 1,2-Butylene Oxide—in Presence of Water

In a 2 l autoclave 146.1 g isosorbide and 15.6 g potassium hydroxide (50% in water) were placed and the reactor heated to 90° C. Vacuum was applied (<25 mbar) and the mixture was heated to 120° C. The mixture was stirred for 2.5 hours at 125° C. and <25 mbar vacuum. The reactor was purged three times with nitrogen. The mixture was heated to 140° C. 721.1 g 1,2-butylene oxide was added within 8 hours. To complete the reaction, the mixture was allowed to post-react for additional 3 hours at 140° C. The reaction mixture was stripped with nitrogen and volatile compounds were removed in vacuo at 90° C. for 2 hours. 865.0 g of a light orange oil was obtained.

¹H-NMR in CDCl₃ confirmed the complete conversion to the expected polymer. Water content was 0.12 wt %.

Step b: Synthesis of Isosorbid, Butoxylated with 24 Mole 1,2-Butylene Oxide and Ethoxylated with 96 Mole Ethylene Oxide

In a 2 l autoclave 104.7 g isosorbid, butoxylated with 10 mole of 1,2-butylene oxide (from step a) were placed. Reactor was purged 3 times with nitrogen. The mixture was heated to 160° C. 120.4 g butylene oxide was added within 2 hours. To complete the reaction, the mixture was allowed to post-react for 6 hours. Reactor was cooled to 150° C., and 504.4 g ethylene oxide was added within 6 hours. To complete the reaction, the mixture was allowed to post-react for additional 2 hours at 150° C. The reaction mixture was stripped with nitrogen, and 1.5 g phosphoric acid (75% in water) was added for neutralization. Volatile compounds were removed in vacuo at 90° C. for 2 hours. 0.10 g alpha-tocopherol was added and the mixture was stirred for 0.25 hours. After cooling, 719.0 g of a beige solid was obtained.

¹H-NMR in CDCl₃ confirmed the complete conversion to the expected polymer. Hydroxy value was measured to be 24.8 mg KOH/g.

### C. EXPERIMENTAL RESULTS

### C.1 Exemplary Block Copolymers (Butronics)

TABLE 1
Exemplary Butronics according to the invention (Test samples No. 1 to 17); all polymers do not
show hemolysis (<10% hemolysis at 100 g/l)

| | | | | | | | | | | wt % | | | | surface |
|---|---|---|---|---|---|---|---|---|---|---|---|---|---|---|
| | | | | | | | | | wt % | EO | | | Cloud | tension |
| | Synthesis | | | | | | | Mw calc | EO¹⁾ | NMR | OH value | water | point | 0.1 g/l |
| | Method | X | R1 | R2 | R3 | m | n | g/mol | % | % | mgKOH/g | solubility | ° C. | mN/m |
| 1²⁾ | A | 1,4-butane diol | Et | H | H | 10 | 17 | 3030 | 50 | 52.6 | 37 | ≥6 wt % | 82 | 37.2 |
| 2 | A | 1,4-butane diol | Et | H | H | 10 | 27 | 3911 | 61 | 63.4 | 35 | ≥10 wt % | 81 | 31.0 |
| 3 | A | 1,4-butane diol | Et | H | H | 5 | 35 | 3895 | 79 | 81.8 | 29 | ≥10 wt % | >95 | 39.8 |
| 4 | A | 1,4-butane diol | Et | H | H | 16 | 40 | 5922 | 60 | 62.6 | n.a. | ≥10 wt % | 83.5 | 37.0 |
| 5 | A | 1,4-butane diol | Et | H | H | 12 | 48 | 6050 | 70 | 71.8 | 24 | ≥10 wt % | >95 | 36.3 |
| 5a | B | 1,4-butane diol | Et | H | H | 12 | 48 | 6050 | 70 | 70.6 | 24.8 | ≥10 wt % | >95 | 39.9 |
| 6 | A | 1,4-butane diol | Et | H | H | 21 | 54 | 7876 | 60 | 62.4 | 21 | ≥10 wt % | 87 | 50.0 |
| 7 | A | 1,4-butane diol | Et | H | H | 10 | 70 | 7658 | 80 | 82.1 | 16 | ≥10 wt % | >95 | 50.1 |
| 8 | A | 1,2-butane diol | Et | H | H | 10 | 70 | 7658 | 80 | 79.4 | n.a | ≥10 wt % | >95 | 43.1 |
| 9 | A | 1,2-butane diol | Et | H | H | 12 | 48 | 6050 | 70 | 66.3 | n.a | ≥10 wt % | 92 | 39.4 |
| 10 | A | 1,2-butane diol | Et | H | H | 10 | 27 | 3911 | 61 | 59.0 | n.a | ≥10 wt % | 74 | 37.8 |
| 11 | A | isosorbid | Et | H | H | 11 | 68 | 7724 | 78 | 79.5 | 15 | ≥10 wt % | >95 | 46.6 |
| 12 | C | isosorbid | Et | H | H | 12 | 48 | 6106 | 69 | 71.9 | 22 | ≥10 wt % | n.a. | 37.7 |
| 13 | C | isosorbid | Et | H | H | 16 | 40 | 5977 | 58 | 60.3 | 22 | ≥10 wt % | n.a. | 44.1 |
| 14 | C | isosorbid | Et | H | H | 12 | 58 | 6987 | 73 | 74.6 | 20 | ≥10 wt % | n.a. | 40.8 |
| 15 | A | 1,6-hexane diol | Et | H | H | 12 | 48 | 6078 | 70 | 70.0 | 22 | ≥10 wt % | n.a. | 35.4 |
| 16 | A | 1,6-hexane diol | Et | H | H | 13 | 58 | 7125 | 71 | 72.6 | 19 | ≥10 wt % | n.a. | 41.1 |
| 17 | A | Neopentylglycol | Et | H | H | 12 | 48 | 6064 | 70 | 68.8 | 21 | ≥10 wt % | n.a. | 42.5 |

¹⁾calculated from the atomic masses of all atoms of the copolymer molecule of formula 1: [Σ masses EO/(Σ masses EO + Σ masses BuO + mass Starter X)] * 100
²⁾not an example of invention (wt.-% EO = 50%)

TABLE 2
Comparative example; all polymers show hemolysis (except for CE5) with >10% hemolysis at 10 g/l)

| | | | | | | | | | | | surface |
|---|---|---|---|---|---|---|---|---|---|---|---|
| | | | | | | | Mw | | | Cloud | tension |
| | | | | | | | calc | wt % EO | water | point | 0.1 g/l |
| | X | R1 | R2 | R3 | m | n | g/mol | % | solubility | ° C. | mN/m |
| CE1 | 1,4-butane | Et | H | H | 5.5 | 7 | 1500 | 40 | ≥10 wt % | 40 | 30.2 |
| | diol | | | | | | | | | | |
| CE2 | 1,4-butane | Et | H | H | 4.5 | 8.5 | 1488 | 50 | ≥10 wt % | 54 | 37.1 |
| | diol | | | | | | | | | | |
| CE3 | 1,4-butane | Et | H | H | 6.5 | 11.5 | 2041 | 50 | ≥10 wt % | 82.5 | 26.7 |
| | diol | | | | | | | | | | |
| CE4 | 1,4-butane | Et | H | H | 5 | 13.5 | 2001.0 | 59 | ≥10 wt % | 78.5 | 31.7 |
| | diol | | | | | | | | | | |
| CE5 | 1,4-butane | Et | H | H | 13 | 23 | 4100 | 50 | <2% | 64 | 41.7 |
| | diol | | | | | | | | | | |

### C.2 Results of the IgG Aggregation Assay

The aggregation tendency assay for the selected set of samples was performed as described above (see item A.4).

For every test three references are tested in addition to the samples: IgG without surfactant (blank), Polysorbate 20 (positive control), and P188 (negative control).

The results are summarized in Table 3.

TABLE 3
Outcome of the aggregation test depicted in
item A.4 for Butronics according to the
invention (Test samples No. 1 to 17)
and comparative Butronics (CE1 to CE5)

| Test samples No. | Aggregation |
|---|---|
| Polysorbate 20 | 6% |
| (positive control) | |
| P188 | 63% |
| (negative control) | |
| 1 | 3% |
| 2 | 3% |
| 3 | 8% |
| 4 | 3% |
| 5 | 2% |
| 6 | 8% |
| 7 | 2% |
| 12 | 2% |
| 13 | 2% |
| 14 | 2% |
| 15 | 1% |
| 17 | 2% |
| CE1 | 2% |
| CE2 | 2% |
| CE3 | 5% |
| CE4 | 3% |
| CE5 | 4% |

As it can be observed all samples inclusive comparative Butronics CE1 to CE5 prevent significant protein aggregation. In that regard, samples 1, 2, 4, 5 and 7, 12 to 15 and 17 proved to prevent protein aggregation to a higher extent than commonly used polysorbate 20.

### C.3 Results of the Water Solubility Assay

The water solubility assay for the selected set of samples was performed as described above (see item A.5).

The results are summarized in Table 4

TABLE 4
Outcome of the water solubility test
Butronics according to the invention
(Test samples No. 1 to 7) and
comparative Butronics (CE1 to CE5)

| | Solubility |
|---|---|
| Test samples No. | in water |
| Kolliphor EL (Ester) | ≥10% |
| Solutol HS15 (Ester) | ≥10% |
| Polysorbate 80 | ≥10% |
| Poloxamer P188 | ≥10% |
| 1 | 6% |
| 2 | ≥10% |
| 3 | ≥10% |
| 4 | ≥10% |
| 5 | ≥10% |
| 6 | ≥10% |
| 7 | ≥10% |
| CE1 | ≥10% |
| CE2 | ≥10% |
| CE3 | ≥10% |
| CE4 | ≥10% |
| CE5 | 1% |

Kolliphor® EL is the registered trademark of polyethoxylated castor oil. It is prepared by reacting 35 moles of ethylene oxide with each mole of castor oil. Kolliphor EL is a synthetic, nonionic surfactant used to stabilize emulsions of nonpolar materials in water. Kolliphor® EL is an excipient or additive used in drugs.

Solutol® HS 15 is the registered trademark of a polyoxyethylated 12-hydroxystearic acid. It is another excipient or additive used in drugs.

As it can be observed, all test samples with exception of test samples 1 and CE5 showed a water solubility of more than 10%. Due to their poor water solubility CE5 is thus unsuitable as formulation excipient.

### C.4 Results of the Surface Tension Assay

The surface tension assay for the selected set of samples was performed as described above (see item A.6).

The results are summarized in Table 5.

TABLE 5
Outcome of the surface tension assay
test depicted in item A.6 for Butronics
according to the invention
(Test samples No. 1 to 10)
and comparative
Butronics (CE1 to CE5)

| | surface |
|---|---|
| | tension |
| | mN/m |
| Test samples No. | (0.1 g/l) |
| Polysorbate 20 | |
| (positive control) | |
| P188 | 52.2 |
| (negative control) | |
| 1 | 37.2 |
| 2 | 31.0 |
| 3 | 39.8 |
| 4 | 37.0 |
| 5 | 36.3 |
| 6 | 50.0 |
| 7 | 50.1 |
| 8 | 43.1 |
| 9 | 39.4 |
| 10 | 37.8 |
| CE1 | 30.2 |
| CE2 | 37.1 |
| CE3 | 26.7 |
| CE4 | 31.7 |
| CE5 | 41.7 |

As it can be observed, all test samples showed surface tension values comprised between 53-30 mN/m (0.1 g/l).

### C.5 Results of the Hemolysis Assay

Hemolysis refers to a phenomenon leading to rupture and dissolution of red blood cells. The assay for the selected set of samples was performed as described above (see item A.7).

The results are summarized in Table 6.

TABLE 6
Outcome of the hemolysis test
depicted in item A.7 for Butronics
according to the invention
(Test samples No. 1 to 7)
and comparative
Butronics (CE1 to CE5)

| | % hemolysis |
|---|---|
| | (av of 2 donors |
| Test samples No. | at 100 g/L) |
| Kolliphor EL (Ester) | 0% |
| Solutol HS15 (Ester) | 18% |
| Polysorbate 80 | 68% |
| Poloxamer P188 | 1% |
| 1 | 0% |
| 2 | 0% |
| 3 | 0% |
| 4 | 0% |
| 5 | 0% |
| 6 | 0% |
| 7 | 0% |
| CE1 | 103% |
| CE2 | 98% |
| CE3 | 82% |
| CE4 | 79% |
| CE5 | 0% |

While CE1 to 4 showed a high degree of hemolysis, test samples 1 to 7, and CE 5 showed a significantly lower haemolytic activity as compared to Polysorbate 80 which is indicative of their low toxicity and thus suitability as possible formulation additives.

### C.6 Small Molecule Solubilisation Ability

The suitability of the Butronics according to the present invention for the solubilisation of model small molecules was tested as described above (see item A.8). The results are summarized in Table 7.

TABLE 7
Outcome of the molecule
solubilisation ability test
depicted in item A.8 for Butronics
according to the invention
(Test samples No. 1 to 7)
and comparative
Butronics (CE1 to CE5)

| | Solubilization |
|---|---|
| | of small |
| | molecules |
| Test samples No. | (mean value in %) |
| Kolliphor EL (Ester) | 100% |
| Solutol HS15 (Ester) | 93% |
| Polysorbate 80 | 96% |
| Poloxamer P188 | 1% |
| 1 | 102% |
| 2 | 87% |
| 3 | 31% |
| 4 | 107% |
| 5 | 82% |
| 6 | 120% |
| 7 | 51% |
| CE1 | 110% |
| CE2 | 96% |
| CE3 | 98% |
| CE4 | 70% |
| CE5 | 126% |

Test samples 4 and 6 and comparative copolymers CE1 and CE5 displayed a superior small molecule solubilizing effect than Kolliphor® EL (reference substance).

C.7 Results of the Preparation of PLGA Nanoparticles with Butronics

PLGA particles were prepared with the different surfactants as described above (see item A.10). The stabilizing properties of the butronics was compared to the stability of the particles without surfactant (only water) and poloxamer P188.

The results are summarized in Table 8.

TABLE 8
The stabilizing properties of the butronics on PLGA nanoparticles

| | Before centrifugation | | After centrifugation | |
|---|---|---|---|---|
| | Particle size | | Particle size | |
| | in nm | | in nm | |
| Surfactant | (by Intensity) | Pdl | (by Intensity) | Pdl |
| without | 228 | 0.10 | Pellet does not resuspend | |
| (only water) | | | | |
| P188 | 251 | 0.09 | Pellet does not resuspend | |
| 1 | 235 | 0.14 | Pellet does not resuspend | |
| 2 | 243 | 0.12 | 235 | 0.08 |
| 3 | 232 | 0.11 | 234 | 0.09 |
| 7 | 252 | 0.10 | Pellet does not resuspend | |
| CE1 | 227 | 0.09 | Pellet does not resuspend | |
| CE2 | 228 | 0.09 | Pellet does not resuspend | |
| CE5 | 241 | 0.14 | 234 | 0.11 |

As it can be observed, test samples 2 and 3 stabilized PLGA particles the best. CE5 was also able to stabilize the particles, but due to their poor water solubility CE5 is less suitable as formulation excipient compared to test samples 2 and 3. For all other test samples the pellet could not be resuspended after centrifugation, indicating a poor stabilization of the PLGA particles.
PDI=Polydispersity index
DLS=dynamic light scattering
PLGA=poly(lactic-co-glycolic acid)

### C.8 Results of Further Biopolymer Aggregation Assays

### C.8.1 Results for β-Casein

The β-Casein aggregation assay for the selected set of samples was performed as described in item A.11.1.

For every test, three references are tested in addition to the samples: β-Casein without surfactant (blank=100%), Polysorbate 20 (positive control), and P188 (negative control).

The results are summarized in Table 9.

TABLE 9
Outcome of the aggregation
assay depicted in item
A.11.1 for Butronics
according to the invention
(Test samples No. 1 to 5)

| Test samples No. | Aggregation |
|---|---|
| Polysorbate 20 | 4% |
| (positive control) | |
| P188 | 29% |
| (negative control) | |
| 1 | 4% |
| 2 | 3% |
| 3 | 4% |
| 4 | 4% |
| 5 | 4% |

As it can be observed, all samples prevent significant protein aggregation. In that regard, samples 1 to 5 proved to prevent protein aggregation to the same extent as commonly used polysorbate 20.

### C.8.2 Results for Lysozyme

The Lysozyme aggregation assay for the selected set of samples was performed as described in item A. 11.2.

For every test, three references are tested in addition to the samples: Lysozyme without surfactant (blank=100%), Polysorbate 20 (positive control), and P188 (negative control).

The results are summarized in Table 10.

TABLE 10
Outcome of the aggregation
assay depicted in item
A.11.2 for Butronics
according to the invention
(Test samples No. 1 to 9)

| Test samples No. | Aggregation |
|---|---|
| Polysorbate 20 | 12% |
| (positive control) | |
| P188 | 62% |
| (negative control) | |
| 1 | 17% |
| 2 | 12% |
| 3 | 12% |
| 4 | 3% |
| 5 | 10% |
| 6 | 10% |
| 7 | 11% |
| 8 | 15% |
| 9 | 6% |

As it can be observed, all samples prevent significant protein aggregation.

### C.8.3 Results for Thrombin

The Thrombin aggregation assay for the selected set of samples was performed as described in item A.11.3.

For every test, three references are tested in addition to the samples: Thrombin without surfactant (blank=100%), Polysorbate 20 (positive control), and P188 (negative control).

The results are summarized in Table 11.

TABLE 11
Outcome of the aggregation
assay depicted in item
A.11.3 for Butronics
according to the invention
(Test samples No. 1 to 9)

| Test samples No. | Aggregation |
|---|---|
| Polysorbate 20 | 16% |
| (positive control) | |
| P188 | 37% |
| (negative control) | |
| 1 | 1% |
| 2 | 1% |
| 3 | 1% |
| 4 | 3% |
| 5 | 3% |
| 6 | 1% |
| 7 | 1% |
| 8 | 1% |
| 9 | 3% |

As it can be observed, all samples prevent significant protein aggregation. In that regard, all Butronics of samples 1-9 proved to prevent protein aggregation to a higher extent as commonly used polysorbate 20.

### C.8.4 Results for Carbonic Anhydrase

The carbonic anhydrase aggregation assay for the selected set of samples was performed as described in item A. 11.4.

For every test, three references are tested in addition to the samples: Thrombin without surfactant (blank=100%), Polysorbate 20 (positive control), and P188 (negative control).

The results are summarized in Table 12.

TABLE 12
Outcome of the aggregation
assay depicted in item
A.11.4for Butronics
according to the invention
(Test samples No. 1 to 8)

| Test samples No. | Aggregation |
|---|---|
| Polysorbate 20 | 518% |
| (positive control) | |
| P188 | 51% |
| (negative control) | |
| 1 | 236% |
| 2 | 161% |
| 3 | 56% |
| 5 | 110% |
| 6 | 190% |
| 7 | 41% |
| 8 | 48% |

Samples 1 to 9 prevent protein aggregation more efficient than polysorbate 20. A high ethylene oxide content (of about 80%) significantly prevents aggregation of Carboanhydrase during shaking.

C.8.5 Results for IgG Extracted from Human Serum

The IgG from human serum aggregation assay for the selected set of samples was performed as described in item A.11.5.

For every test, three references are tested in addition to the samples: Thrombin without surfactant (blank=100%), Polysorbate 20 (positive control), and P188 (negative control).

The results are summarized in Table 13.

TABLE 13
Outcome of the aggregation
assay depicted in item
A.11.5 for Butronics
according to the invention
(Test samples No. 1 to 9)

| Test samples No. | Aggregation |
|---|---|
| Polysorbate 20 | 243% |
| (positive control) | |
| P188 | 90% |
| (negative control) | |
| 1 | 9% |
| 2 | 2% |
| 3 | 11% |
| 4 | 4% |
| 5 | 7% |
| 6 | 16% |
| 7 | 6% |
| 8 | 8% |
| 9 | 4% |

As it can be observed, samples 1-9 prevent significant protein aggregation.

C.8.6 Results for Monoclonal Antibody 4 (mAb4)

The mAb4 assay for the selected set of samples was performed as described in item A.11.6.

For every test set, four references are tested in addition to the samples: mAb4 without surfactant and stored at room temperature without shaking (unstressed mAb4), mAb4 without surfactant and stressed by shaking (blank=100%), mAb4 with PS20 (positive control), and mAb4 with P188 (negative control).

Since the blank samples are very turbid, those samples are not further analyzed by micro-flow imaging. For analysis, the particle count of the samples is set in relation to the aggregation of the unstressed mAb4 (the particle count of the unstressed mAb4 is set as 100% aggregation).

The results are summarized in Table 14.

TABLE 14
Outcome of the
aggregation assay depicted
in item A. 11.6 for Butronics
according to the invention
(Test samples No. 1, 2, 4, 5, 7- 9)

| Test samples No. | Aggregation |
|---|---|
| Polysorbate 20 | 1751% |
| (positive control) | |
| P188 | 451% |
| (negative control) | |
| 1 | 237% |
| 2 | 122% |
| 4 | 123% |
| 5 | 91% |
| 7 | 178% |
| 8 | 201% |
| 9 | 378% |

All samples significantly prevent mAb4 aggregation.

C.8.7 Results for Monoclonal Antibody 3 (mAb3)

The mAb3 assay for the selected set of samples was performed as described in item A.11.7.

For every test set, four references are tested in addition to the samples: mAb3 without surfactant and stored at room temperature without shaking (unstressed mAb3), mAb3 without surfactant and stressed by shaking (blank=100%), mAb3 with PS20 (positive control), and mAb3 with P188 (negative control).

The results are summarized in Table 15.

TABLE 15
Outcome of the
aggregation assay depicted
in item A.11.7 for Butronics
according to the invention
(Test samples No. 1 to 9)

| Test samples No. | Aggregation |
|---|---|
| Polysorbate 20 | 42% |
| (positive control) | |
| P188 | 8% |
| (negative control) | |
| unstressed mAb3 | 6% |
| 1 | 20% |
| 2 | 10% |
| 3 | 5% |
| 4 | 6% |
| 5 | 4% |
| 6 | 5% |
| 7 | 4% |
| 8 | 4% |
| 9 | 7% |

As it can be observed, all samples prevent significant protein aggregation.

### C.9 Summary

Our attempts to provide polymeric surfactants as superior alternative to Polysorbates 20/80 and Poloxamer 188 allow the following conclusions:

For polyclonal IgG, all tested samples (samples 1 to 17 and 5a) proved to be good stabilizing agents against aggregation for protein formulations.

Looking at the stabilization performance of butronics with biopolymers especially samples 4, 5, 7, 8, 9 show outstanding performance and prevented protein aggregation to a higher extent than commonly used polysorbate 20.

Sample 5, 7, 8 and 9 are the best performing samples in the assays tested here. Said samples are also characterized by suitable surface tension (~within the range of 53-30 mN/m (0.1 g/l)) and molecular weight (with Mw about 6.000-8.000 g/mol). Sample 1 and CE5 proved to be lower in water solubility, whereas comparative butronics CE1 to CE4 showed a high degree of hemolysis.

Accordingly, samples 5, 7, 8 and 9 perform best as regards all criteria necessary for employment as surfactant for biopolymer formulations with superior properties in comparison to Polysorbates 20/80 and Poloxamer 188.

Furthermore, samples 2, 4, 5, 7, 8 and 9 perform best as regards all criteria necessary for the employment as surfactant for antibody formulations with superior properties in comparison to Polysorbate 20/80 and Poloxamer 188.

Additionally, samples 4 and 6, were shown to be characterized by a particularly good small molecules solubilizing effect which makes them particularly suitable surfactants for ADCs formulations.

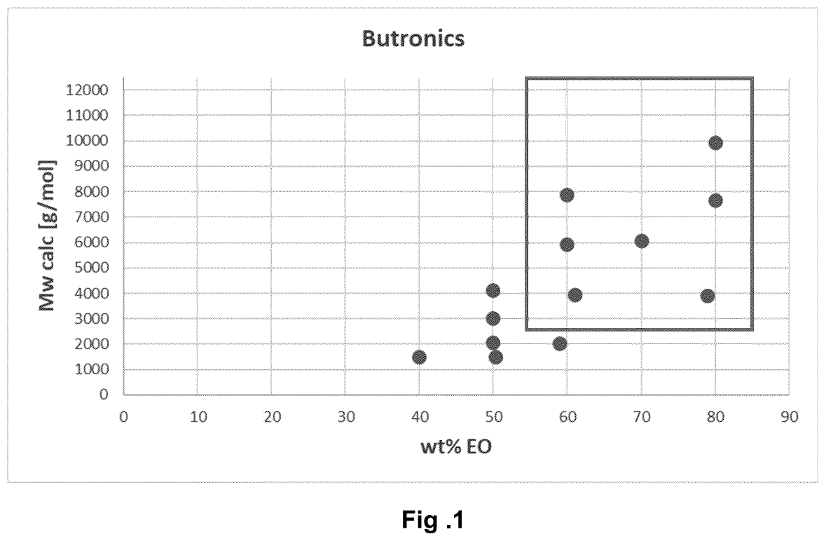

The surprising advantages associated with particular examples of butronics of the present invention are summarized in attached FIG. 1.

The content of all documents referred to herein above is incorporated by reference.

1.-19. (canceled)

20. A biopolymer solution, comprising an aqueous solution, of at least one biopolymer component and at least one ethylene oxide/butylene oxide block copolymer of the general formula 1 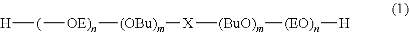
in which
X represents —O— or a divalent organic moiety;
m independently of each other represents an integer in the range of 4 to 25; and
n independently of each other represents an integer in the range of 15 to 100;
wherein
said biopolymer is selected from oligopeptides, polypeptides, proteins, antibody molecules or fragments or derivatives thereof, glycosylated proteins, proteoglycans, oligo- and polynucleotides, DNA and RNA molecules, oligosaccharides, polysaccharides, as well as adducts or conjugates of such biopolymers with a further constituent selected from payload molecules;
and
said block copolymer shows a combination of the following features:
a) a molecular weight of 2.500 to 12.500 g/mol, calculable from the sum of atomic masses of all atoms of the copolymer molecule of general formula 1;
b) an EO content of >50 to 85 wt.-%, based on the dry weight of said block copolymer, calculable from the atomic masses of all atoms of the copolymer molecule of formula 1;
c) a water solubility of at least 5 wt.-%, based on the total weight of the aqueous solution of the block copolymer; and
d) less than 10% hemolytic activity caused by a solution of said block copolymer of 100 g/l.

21. The biopolymer solution of claim 20, wherein said block copolymer shows at least one of the following additional features
a) a water solubility of at least 10 wt.-%, based on the total weight of the aqueous solution of the block copolymer;
b) a surface tension SFT of 53 to 30 mN/m based on a 0.1 g/l solution of said block copolymer; and
c) a lack of hemolytic activity.

22. The biopolymer solution of claim 20, wherein the butylene oxide block is composed of monomer units derived from 1,2-butylene oxide, 2,3-butylene oxide, isobutylene oxide, or mixtures thereof, or
wherein
X is selected from —O— and —O-alkylene-O—, wherein the alkylene chain is straight-chained or branched, and is optionally interrupted by one or more heteroatoms; or X is a group of the formula 2

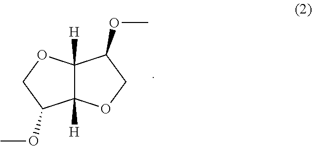

23. The biopolymer solution of claim 20, wherein said block copolymer of general formula 1 shows a combination of the following features
a) a molecular weight 5.500 to 8.000 g/mol, calculable from the sum of atomic masses of all atoms of the copolymer molecule of general formula 1;
b) an EO content of 60 to 85 wt.-%, based on the dry weight of said block copolymer, calculable from the atomic masses of all atoms of the copolymer molecule of formula 1; and
c) X=—O-n-butylene-O—.

24. The biopolymer solution of claim 20, wherein the block copolymer is selected from the following compounds of the general formula 1, wherein X, m and n have the following meanings:
X=—O-n-butylene-O—, m=10; and n=17;
X=—O-n-butylene-O—, m=10, and n=27;
X=—O-n-butylene-O—, m=5, and n=35;
X=—O-n-butylene-O—, m=16, and n=40;
X=—O-n-butylene-O—, m=12, and n=48;
X=—O-n-butylene-O—, m=21, and n=54;
X=—O-n-butylene-O—, m=10, and n=70;
X=a moiety of formula 2, m=10; and n=17;
X=a moiety of formula 2, m=10, and n=27;
X=a moiety of formula 2, m=5, and n=35;
X=a moiety of formula 2, m=16, and n=40;
X=a moiety of formula 2, m=12, and n=48;
X=a moiety of formula 2, m=21, and n=54;
X=a moiety of formula 2, m=11, and n=68; or
X=a moiety of formula 2, m=10, and n=70.

25. The biopolymer solution of claim 24, wherein the block copolymer is selected from the following compounds of the general formula 1, wherein X, m and n have the following meanings:
X=—O-n-butylene-O—, m=12, and n=48 and a molecular weight of approximately 6.050, calculable from the sum of atomic masses of all atoms of the copolymer molecule of general formula 1;
X=—O-n-butylene-O—, m=10, and n=70 and a molecular weight of approximately 7.658, calculable from the sum of atomic masses of all atoms of the copolymer molecule of general formula 1;
X=—O-n-butylene-O—, m=16, and n=40 and a molecular weight of approximately 5.922, calculable from the sum of atomic masses of all atoms of the copolymer molecule of general formula 1; or
X=a moiety of formula 2, m=11, and n=68 and a molecular weight of approximately 7.724, calculable from the sum of atomic masses of all atoms of the copolymer molecule of general formula 1.

26. The biopolymer solution of claim 22, wherein or X is a group of the formula 2 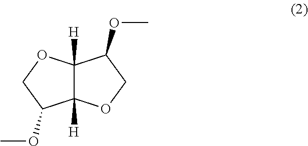
wherein
said block copolymer shows a combination of the following features
a) a molecular weight of 3.000 to 8.000 g/mol, calculable from the sum of atomic masses of all atoms of the copolymer molecule of general formula 1;
b) an EO content of 55 to 85 wt.-% based on the dry weight of said block copolymer, calculable from the atomic masses of all atoms of the copolymer molecule of general formula 1;
c) a water solubility of at least 5 wt.-% based on the total weight of the aqueous solution of the block copolymer; and
d) less than 10% hemolytic activity caused by a solution of 100 g/l.

27. The biopolymer solution of claim 20, wherein the biopolymer is selected from oligopeptides, polypeptides, proteins, glycosylated proteins, proteoglycans, antibody molecules or fragments or derivatives thereof, adducts or conjugates of such biopolymers with a further constituent selected from payload molecules, selected from
a) pharmaceutically active compounds;
b) labeling agents;
c) biological small molecules such as lipids, phospholipids, glycolipids, sterols, vitamins, hormones, neurotransmitters, amino acids, nucleotides, monosaccharides; and
d) biological macromolecules, such as peptides, oligopeptides, polypeptides, proteins, nucleic acids, such as any forms of DNA and RNA, oligosaccharides, and polysaccharides.

28. The biopolymer solution of claim 27, wherein said biopolymer is a diagnostically applicable or a therapeutically active biopolymer; or
said biopolymer is selected from adducts or conjugates of an immunoglobulin molecule and a payload molecule; or
said biopolymer is an antibody payload conjugate (APC), particularly an antibody drug conjugate (ADC).

29. The biopolymer solution of claim 20, wherein said biopolymer is an immunoglobulin or protein molecule or an antibody payload conjugate (APC), each optionally glycosylated, wherein said at least one ethylene oxide/butylene oxide block copolymer is of the general formula 1, 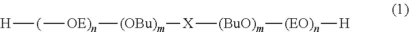
in which
X represents —O—; or a divalent organic moiety;
m independently of each other represents an integer in the range of 10 to 20; and
n independently of each other represents an integer in the range of 25 to 75;
wherein said block copolymer shows a combination of the following features:
a) a molecular weight of 3.500 to 8.000 g/mol, calculable from the sum of atomic masses of all atoms of the copolymer molecule of general formula 1;
b) an EO content of 55 to 85 wt.-%, based on the dry weight of said block copolymer, calculable from the atomic masses of all atoms of the copolymer molecule of general formula 1;
c) a water solubility of at least 5 wt.-% based on the total weight of the aqueous solution of the block copolymer; and
d) less than 10% hemolytic activity caused by a solution of 100 g/l.

30. The biopolymer solution of claim 20, wherein said block copolymer of the general formula 1 is contained in a proportion of 0.001 to 10%, based on the total weight of the biopolymer solution; or said biopolymer is contained in a proportion of 0.01 to 30%, based on the total weight of the biopolymer solution; or wherein said biopolymer solution is in optionally buffered form, having a pH in the range of 5 to 9.

31. An essentially dry biopolymer composition, comprising at least one biopolymer component as defined above, and at least one ethylene oxide/butylene oxide block copolymer of the general formula 1 as defined in claim 20; which has a liquid content of 0% to 5% wt.-% based on the total weight of said composition;
and is optionally further characterized as follows:
wherein said at least one block copolymer (A) and said at least one biopolymer (B) are contained in a weight ratio (A):(B) in the range of 1:20.000 to 10:1, or 1:5.000 to 2:1, 1:100 to 1.2:1;
or
wherein said at least one block copolymer and said at least one biopolymer together are contained in a proportion of 1 to less than 100 wt.-%, based on the total weight of said essentially dry composition;
or
further comprises at least one further excipient in a proportion of 0.1 to 99 wt.-%, 40 to 95 wt.-% and 50 to 90 wt.-% based on the total dry weight of said essentially dry composition.

32. The composition of claim 20 for use in medicine, in particular for diagnostic or therapeutic applications.

33. The composition of claim 20, which is a pharmaceutical composition, optionally further supplemented by at least one pharmaceutically acceptable excipient.

34. A method of preparing a stabilized biopolymer solution of claim 20, which method comprises
a) preparing in any order an aqueous, optionally buffered solution of the biopolymer; and an aqueous, optionally buffered solution of the block copolymer of general formula 1, and
b) preparing a mixture of both aqueous solutions as obtained in step a).

35. A method of preparing the essentially dry stabilized biopolymer solution of claim 31, which method comprises
a) preparing in any order an aqueous, optionally buffered solution of said biopolymer; and an aqueous, optionally buffered solution of said block copolymer of general formula 1;
b) preparing a mixture of both aqueous, optionally buffered, solutions as obtained in step a);
c) optionally supplementing the aqueous, optionally buffered solutions prepared in step a) or the mixture of both aqueous, optionally buffered, solutions prepared in step b), with at least one pharmaceutically acceptable excipient; and
d) drying the mixture obtained in step b) or c).

36. A block copolymer selected from the following compounds of the general formula 1, wherein X, m and n have the following meanings:
X=—O-n-butylene-O—, m=10; and n=17;
X=—O-n-butylene-O—, m=10, and n=27;
X=—O-n-butylene-O—, m=5, and n=35;
X=—O-n-butylene-O—, m=16, and n=40;
X=—O-n-butylene-O—, m=12, and n=48;
X=—O-n-butylene-O—, m=21, and n=54;
X=—O-n-butylene-O—, m=10, and n=70;
X=a moiety of formula 2, m=10; and n=17;
X=a moiety of formula 2, m=10, and n=27;
X=a moiety of formula 2, m=5, and n=35;
X=a moiety of formula 2, m=16, and n=40;
X=a moiety of formula 2, m=12, and n=48;
X=a moiety of formula 2, m=21, and n=54;
X=a moiety of formula 2, m=11, and n=68; or
X=a moiety of formula 2, m=10, and n=70.

37. The block copolymer of claim 36, wherein X, m and n have the following meanings:
X=—O-n-butylene-O—, m=12, and n=48 and a molecular weight of approximately 6.050, calculable from the sum of atomic masses of all atoms of the copolymer molecule of general formula 1;
X=—O-n-butylene-O—, m=10, and n=70 and a molecular weight of approximately 7.658, calculable from the sum of atomic masses of all atoms of the copolymer molecule of general formula 1;
X=—O-n-butylene-O—, m=16, and n=40 and a molecular weight of approximately 5.922, calculable from the sum of atomic masses of all atoms of the copolymer molecule of general formula 1; or
X=a moiety of formula 2, m=11, and n=68 and a molecular weight of approximately 7.724, calculable from the sum of atomic masses of all atoms of the copolymer molecule of general formula 1.
